(19) United States
(12) Patent Application Publication
(10) Pub. No.: US 2024/0212855 A1
(43) Pub. Date: Jun. 27, 2024

(54) SYSTEMS, METHODS AND WEARABLE BIOSENSING DEVICES FOR USE IN A DIAGNOSTIC ARCHITECTURE

(71) Applicant: Alio, Inc, Broomfield, CO (US)

(72) Inventors: Samir Shreim, Lake Forest, CA (US); David Whittaker, Potomac, MD (US); Forrest Miller, Boston, MA (US); Aamani Budhota, Edinburgh (GB); Samit Gupta, Boulder, CO (US)

(21) Appl. No.: 18/391,402

(22) Filed: Dec. 20, 2023

Related U.S. Application Data
(60) Provisional application No. 63/434,577, filed on Dec. 22, 2022.

(51) Int. Cl. G16H 50/30; A61B 5/00; A61B 5/024; G16H 40/67; G16H 50/20; G16H 50/70

(52) U.S. Cl. CPC G16H 50/30; A61B 5/02416; A61B 5/7275; A61B 5/742; G16H 40/67; G16H 50/20; G16H 50/70

(57) ABSTRACT

A system and method are disclosed that include a wearable biosensing device mounted over or proximate to a vessel of a patient enabling biosensing data to be obtained or captured by the wearable biosensing device. Operations of the method or performed by logic of the system are directed at determining patient health-related predictions of a first patient and include obtaining patient health data including at least biosensing data obtained from a wearable biosensing device of the first patient, wherein the biosensing data includes one or more of raw signals captured by the wearable biosensing device or a first metric provided by the wearable biosensing device, evaluating the patient health data by applying one or more machine learning techniques that receive the patient health data as input resulting in a first patient health-related prediction, and generating a graphical user interface illustrating the first patient health-related prediction.

### PRIORITY

This application claims the benefit of priority to U.S. Provisional Application No. 63/434,577, filed Dec. 22, 2022, which is incorporated by reference in its entirety into this application.

### FIELD

Embodiments of the disclosure relate to the field of wearable biosensing devices and diagnostic analytics resulting from data acquired therefrom. More specifically, one embodiment of the disclosure relates to a closed-loop architecture that includes the capture of data from a wearable biosensing device, integration of the captured biosensing data with data captured by peripheral devices, diagnostic data, and/or patient demographic data, and generation of a treatment recommendation that may be provided directly to a patient.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Over the last decade, there has been an increasing number of wearable biosensing devices. These devices include one or more biosensors, which collect health-related data from a user. Most of these wearable biosensing devices include a display (e.g., smart watches, fitness trackers, etc.) and are mounted on the user's wrist using a band which encircles the wrist. However, these display-based devices are costly, non-disposable, and cannot be targeted to monitor certain health characteristics, such as blood flow for example, at other location on the wearer besides the wrist area.

Additionally, the wearable biosensing devices often provide metrics that are sensed or measured by the wearable biosensing device to the user such as heart rate data, a number of steps over a 24 hour period, a total distance traveled by the wearer over a 24 hour period, and exercise data (time, heart rate data, estimated calories burned, steps, etc.) during a specified period of exercise. However, such metrics merely provide a surface level amount of information to a wearer. In fact, such metrics may also be misleading by giving the wearer a false sense of health by providing the wearer he or she accomplished an arbitrary health goal of standing for 12 hours during a 24 hour period or taking a total of 6,000 steps during a 24 hour period. Such metrics fail to provide an indication as to, for example, blood flow metrics such as pulsatile vascular blood flow and pulsatile vascular expansion, among others.

In particular, abnormal serum potassium levels in patients with long term health conditions including heart failure, diabetes mellitus and chronic kidney disease can result in significant morbidity and mortality due to cardiac arrhythmias and myocardial dysfunction. Patients with advanced or end-stage kidney disease often develop abnormal potassium blood levels, such as hyperkalemia or hypokalemia. Hyperkalemia occurs in about 8-10% of patients receiving hemodialysis. In 24% of hyperkalemic episodes, patients require an emergency hemodialysis session. Screening for electrolyte imbalance is typically carried out through blood draws requiring laboratory testing and evaluation. The ability to repeatedly monitor changes in electrolyte levels daily, without blood draws, could potentially lower health care costs, reduce patient morbidity and inform dietary measures and titration of dialysis regimens to ensure an optimal electrolyte balance is consistently maintained without the need for emergent interventions.

Improvements in dialysis technology have enhanced the opportunity for a broader global population to utilize home dialysis instead of facility-based dialysis. In the United Kingdom, hospitals and health organizations have sought methods to help patients remotely monitor renal function. In the United States, as of 2017, less than 12% of the population participated in home dialysis (whether by peritoneal dialysis or home hemodialysis). Additionally, in the United States, the Advancing American Kidney Health Initiative of 2019 seeks to improve access to home dialysis for the 85% of patients considered eligible for home dialysis.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1A is a block diagram illustrating a first system architecture of a wearable biosensing device coupled to set of components operating within a networked environment in accordance with some embodiments;

FIG. 1B is a block diagram illustrating a second system architecture of a wearable biosensing device coupled to set of components operating within a networked environment in accordance with some embodiments;

FIG. 2 is a flow diagram illustrating interoperability of the components of FIGS. 1A-1B in accordance with some embodiments;

FIG. 3 is an exploded view of an exemplary embodiment of the wearable biosensing device of FIGS. 1A-1B, which includes a first housing, a shielding component positioned under biosensing logic, a second housing, and an adhesive layer in accordance with some embodiments;

FIG. 4 is an exemplary block diagram of the biosensing components of FIG. 3 in accordance with some embodiments;

FIG. 5A is a flow diagram illustrating operations of an example operating system for the wearable biosensing device of FIG. 5A in accordance with some embodiments;

FIG. 5B is a flow diagram illustrating detailed operations comprising the acquisition of sensor modalities and processing sensor data in accordance with some embodiments;

FIG. 6 is a logic diagram illustrating logic modules of a diagnostic and treatment determination system (“system”) stored on non-transitory storage and configured to be executed by one or more processors in accordance with some embodiments;

FIG. 7A is a flow diagram illustrating operations performed by the system of FIG. 6 to train and apply a machine learning model to generate a prediction of the health status of a patient in accordance with some embodiments;

FIG. 7B is a flow diagram illustrating operations performed by the system of FIG. 6 to train and apply a machine learning model to generate a classification of a potassium imbalance within a patient in accordance with some embodiments;

FIG. 7C is a flow diagram illustrating operations performed by the system of FIG. 6 to identify high quality measurements captured by the biosensing device 100 and detection of metrics through the use of machine learning techniques in accordance with some embodiments;

FIGS. 8A-8B is a flow chart illustrating operations performed by the system of FIG. 6 to apply a machine learning model to data obtained by a wearable biosensing device and external devices to generate a prediction of the health status of a patient and personalizing the machine learning model for a particular patient through iterative retraining using data obtained by a biosensing device worn by the patient in accordance with some embodiments;

FIG. 9 is a logical flow diagram illustrating exemplary processing flows within an analytics logic resulting in one or more of a risk score determination, an assessment recommendation, a treatment recommendation for a clinician or patient, and/or executable dialysis machine instructions in accordance with some embodiments;

FIGS. 10A-10B provide an illustration of a graphical user interface configured for display in a web browser and displaying results of analytics performed by the system of FIG. 6 in accordance with some embodiments; and

FIG. 11 provides an illustration of a graphical user interface configured for display via an application processing on a network device and displaying results of analytics performed by the system of FIG. 6 in accordance with some embodiments.

### DETAILED DESCRIPTION

Embodiments of the present disclosure generally relate to a wearable biosensing device and a data processing system. The biosensing device features an operating system that may include or be configured to process logic deployed within a housing that is attached to a wearer through an adhesive for example. The wearable biosensing device includes an electronics assembly, a power assembly, and a sensing assembly positioned between the electronics assembly and the power assembly. The data processing system is configured to obtain biosensing data, diagnostic data, patient data, and/or peripheral data, and perform various analytics thereon resulting in the determination of one or more of a risk scale profile, an assessment recommendation to a clinician, a treatment recommendation to a clinician or a patient, and/or executable machine instructions provided directly to medical equipment such as a dialysis machine.

As discussed herein, biosensing data may include raw signals, constructed indexes, and/or metrics obtained or determined by wearable biosensing device. Biosensing data may also refer to energy measurements, where the energies captured by the biosensing device may include light energy, acoustic energy, and/or mechanical force. Diagnostic data may include blood test results, diabetes diagnosis or other pre-existing medical conditions, etc. Patient data may include gender, weight, age, body mass index, skin color (skin-tone), geographic information, etc. Peripheral data may include metrics sensed by peripheral devices such as a scale or dialysis machine. In some instances, at least a portion of patent data may be obtained from an electronic health record (EHR), which is known in the art to be an electronic version of a patient's medical history. An application programming interface (API) may be utilized by the data processing system 140 to communicate with an EHR to obtain patient data. The communication may be either a pull or push configuration such that the data processing system 140 may transmit requests (queries) to the EHR for patient data of a particular patient (pull configuration) and/or may receive transmissions including patient data of a particular patient from the HER, e.g., upon a change or at predetermined intervals (push configuration).

The data processing system may include one or more machine learning models and/or neural networks (e.g., deep learning neural networks) configured to evaluate the biosensing data, diagnostic data, patient data, and/or peripheral data resulting in one or more patient health-related predictions. Examples of patient health-related predictions include metric data, a patient health status, a clinical recommendation, a risk stratification, etc. Predicted metric data may refer to expected blood flow rates, heart rate, plethysmograph (PPG) and laser speckle plethysmographic (SPG) waveforms, fluid composition within a vessel (e.g., an artery, a vein, or an arteriovenous (AV) fistula), etc. The patient health status may refer generally to an overall health of the patient which may be determined at least in part through correlation of biosensing data, peripheral device data, and diagnostic data. For example, a patient health status may be represented as a numerical score (e.g., 0-100) that is determined relative to the patient's diagnostic data (e.g., diagnosed medical conditions), where a patient that is under/overweight or has poor blood flow is determined to have a lower patient health status score than would otherwise be determined if not for being under/overweight or having poor blood flow. The patient health status provides a patient with a quick reference to their overall health while accounting for several complex metrics. In some instances, an alert may be generated for the patient and/or a clinician when the patient health status falls below a threshold score (e.g., alternative scoring methods may be used such a categorical scoring, e.g., “poor,” “healthy” “great health,” etc.).

A risk stratification may refer to a grouping of a plurality of analyses results. A risk stratification may cover a multiple phases, with each phase referencing a different analysis result or manner of delivery an analysis result. For example, a risk stratification may include multiple phases including phase 1 (risk scale), phase 2 (assessment recommendation), phase 3 (treatment recommendation).

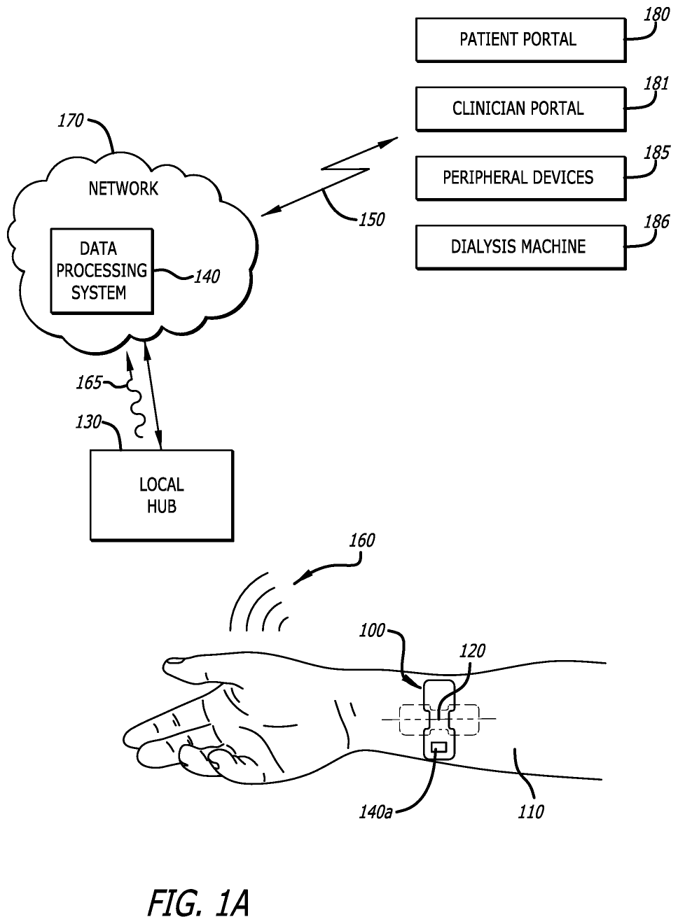

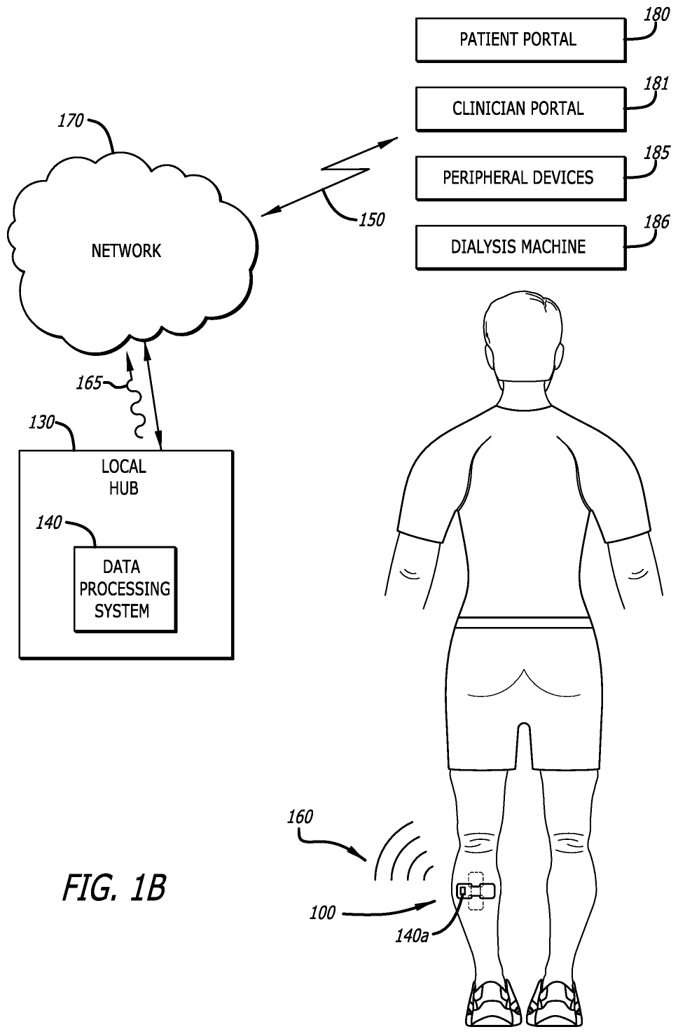

Phase 1 may result in determination of a risk score and/or risk scale. During this phase, biosensing sensing data may be captured by a biosensing device of FIGS. 1A-1B and provided to the data processing system 140. Additional information may also be obtained by the data processing system 140 including peripheral data (e.g. from other devices such as a scale or wearable fitness tracker), diagnostic data (e.g., blood test results, diabetes diagnosis, etc.) and/or patient data (e.g., demographic data, male/female, age, etc.). The data processing system 140 performs processing to generate a risk score (e.g., indicating the risk of kidney failure). As discussed in detail below, the data processing system 140 may deploy one or more machine learning models that take the collected data (as features) as input and based on the weightings of features computed during training of the machine learning model, provide a risk score. The risk score is indicative of the prediction generated by the machine learning model as to the likelihood that the input features indicate kidney failure. It should be understood that a machine learning model may be trained to provide a risk score indicative of other health conditions or health-related metrics.

In some embodiments, the risk score may be provided to the patient or a clinician and used as one piece of information in determining a treatment decision. For instance, a risk scale may be generated based on a plurality of risk scores for a patient over time. Each risk score reflects a patient's health status for a particular measurement or condition (e.g., kidney failure, potassium level, etc.) at a single point in time while the risk scale provides those risk scores over time so that trends may be determined. In some embodiments, the data processing system 140 may deploy an additional machine learning model to detect trends within the risk scale. Alerts may be generated based on the risk score, such as based on a threshold comparison, or on the risk scale, such as in view of a detected trend or a difference between a previous risk score (such as detecting a change above a threshold amount between the current risk score and an immediately preceding risk score, or between the current risk score and any of a risk score determined within a predetermined time period such as within 3-5 days). The alerts may be provided to either the patient or the clinician via the patient portal 180 or the clinician portal 181.

Phase 2 may result in an assessment recommendation. Similar to phase 1, the data processing system 140 collects specific data including biosensing sensing data, peripheral data, diagnostic data, and/or patient data. In some embodiments, analysis by the data processing system 140 may result in a prediction of metric data of an individual wearing the biosensing device 100, where the prediction is compared to a predetermined threshold (e.g., provided by a clinician or otherwise predetermined). Based on the threshold comparison such as not satisfying the threshold comparison (e.g., indicating a low hemoglobin (Hb) level), the data processing system 140 may generate an assessment recommendation that includes an instruction to a clinician to review the medications being prescribed and/or possible medications to prescribe with an indication that adjustment of a current set of prescriptions may be able to achieve improved health metrics, e.g., improved Hb levels, improved potassium levels, etc.

Phase 3 may result in a treatment recommendation. Similar to phases 1-2, the data processing system 140 collects specific data including biosensing sensing data, peripheral data, diagnostic data, and/or patient data. In some embodiments, the data processing system 140 may obtain patient data from an EHR that includes dietary, supplement, prescription, and/or other medication information, which may include dosing information. In such embodiments, features may be extracted from the dietary, supplement, prescription, and/or other medication information and passed as part of an input feature vector to a trained machine learning model, as discussed below. When such machine learning models are trained using historical data that includes such dietary, supplement, prescription, and/or other medication information (with applicable dosing information), the trained machine learning model may provide a prediction as to dosing information of a particular medication (or combination thereof) that would be expected to improve the patient's health. As one illustrative example, the machine learning deployment by the data processing system 140 may result in a prediction of a particular erythropoietin stimulating agents (ESA) medication dosing for a patient in order to increase the patient's Hb levels.

In some examples, a treatment recommendation, which may be received by either the patient, a clinician, and/or a healthcare proxy, may include a recommended dialysis treatment plan (e.g., a suggested time and length for a next dialysis treatment), a recommended medicinal plan (e.g. erythropoietin dosing, frequency, etc.), a recommended nutrition intake plan (e.g., a recommendation of a number of calories or amount of water, fats, carbohydrates, proteins, vitamins, minerals (e.g. iron) etc., to be consumed over a certain time period or at a particular time), a recommendation pertaining to the health of a vessel (e.g., a health of an AV fistula being monitored by the wearable biosensing device).

As noted above with respect to phases 1-2, the treatment recommendation may be provided to either the patient or the clinician via the patient portal 180 or the clinician portal 181. In some instances, following the deployment of the machine learning model(s), the treatment recommendation may be made accessible by either the portals 180, 181 and an alert may be providing directly to a patient or clinician depending on to whom access to the treatment recommendation is provided. The alert may be provided over a wireless network such as via text message or email. Additionally, or alternatively, the alert may comprise a pop-up, banner, badge, or other icon on a graphical user interface of either portal 180, 181.

In some embodiments, the data processing system may also be configured to predict or respond to dialysis parameters, which may be converted into instructions that are configured to by a dialysis machine.

As an illustrative example, the wearable biosensing device (with remote monitoring) may be mounted over or proximate to a vessel, such as an artery, a vein, or an arteriovenous (AV) fistula as disclosed in U.S. Pat. Nos. 11,045,123 and 11,406,274, the contents of both of which are incorporated by reference herein. The AV fistula is a surgical connection made between an artery and a vein, usually created by a vascular specialist. The AV fistula facilitates more efficient dialysis than a “line” port due to quicker blood flow during a dialysis session. The AV fistula is typically located in the arm, however, if necessary, it can be placed in the leg. Other uses for the wearable biosensing device include mounting on the chest for monitoring cardiac functions or on the abdomen for prenatal or intestinal monitoring.

The wearable biosensing device may be configured to obtain an audio pattern (e.g., sound waves) via an audio sensing component (e.g., microphone, etc.) and perform certain actions in response to detection of a particular audio pattern. For example, certain detected audio frequencies and/or audio patterns may be used to identify a change in operability (e.g., flow rate, occlusion of the vessel, etc.) experienced by the physiologic site to which the wearable biosensing device is placed or adhered. The wearable biosensing device may include additional sensing elements such as a PPG and SPG sensors (e.g., a laser and camera module), accelerometer sensors, strain gage and other types of force sensors, acoustic sensor (including those that emit and detect sound energy), etc. and be configured to determine waveforms from the detected raw signals. The data collected by the wearable biosensing device may refer to biosensing data and be provided to the data processing system.

The data processing system may also receive or otherwise obtain data from one or more peripheral devices such as via a software application that interfaces with a bodyweight scale as well as patient data and/or diagnostic data. The data processing system then deploys one or more artificial intelligence techniques to evaluate the obtained data in order to form one or more predictions as to the patient's health. As explained below, these predictions may be indicative of a future change in operability of the AV fistula, an indication as to whether the patient is consuming enough (or too much water), a treatment recommendation such as a recommended dialysis treatment or meal, etc. These predictions may be provided directly to the patient and/or a clinician through a graphical user interface accessible on a network device.

### I. Terminology

In the following description, certain terminology is used to describe aspects of the invention. The terms “logic” or “assembly” are representative of hardware, firmware, and/or software that is configured to perform one or more functions. As hardware, the logic (or assembly) may include circuitry associated with data processing, data storage and/or data communications. Examples of such circuitry may include, but are not limited or restricted to a processor, a programmable gate array, a microcontroller, an application specific integrated circuit, wireless receiver, transmitter and/or transceiver circuitry, sensors, semiconductor memory, and/or combinatorial logic.

Alternatively, or in combination with the hardware circuitry described above, the logic (or assembly) may include software in the form of one or more software modules (hereinafter, “software module(s)”), which may be configured to support certain functionality upon execution by data processing circuitry. For instance, a software module may constitute an executable application, daemon application, application programming interface (API), subroutine, function, procedure, applet, servlet, routine, source code, shared library/dynamic load library, or one or more instructions. The “software module(s)” may be stored in any type of a suitable non-transitory storage medium, or transitory storage medium (e.g., electrical, optical, acoustical, or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, a hard disk drive, an optical disc drive, a portable memory device, or cloud-based storage (e.g., AWS S3 storage), etc. As firmware, the logic (or assembly) may be stored in persistent storage.

The terms “member” and “element” may be construed as a hardware-based logic. The term “attach” and other tenses of the term (e.g., attached, attaching, etc.) may be construed as physically connecting a first member to a second member.

The term “interconnect” may be construed as a physical or logical communication path between two or more components such as a pair of assemblies. For instance, as a physical communication path, wired interconnects in the form of electrical wiring, optical fiber, cable, and/or bus trace. As a logical communication path, the interconnect may be a wireless channel using short range signaling (e.g., BLUETOOTH®) or longer range signaling (e.g., infrared, radio frequency “RF” or the like).

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. General Architecture Schemes

Referring to FIG. 1A, a first system architecture of a wearable biosensing device coupled to set of components operating within a networked environment is shown in accordance with some embodiments. The wearable biosensing device 100 is intended to be worn over a vessel 120 (e.g., an artery, a vein, or an arteriovenous (AV) fistula) of a patient 110 and configured to direct collected information (“biosensing data”) to a remotely located, data processing system 140. For this embodiment, the wearable biosensing device 100 is configured to monitor properties (e.g., characteristics and operability) of the AV fistula 120 by collecting information associated with the AV fistula 120 and the biological fluid propagating therethrough (e.g., flow, fluid composition inclusive of Hb levels, etc.). The collected information may be used for remote monitoring, where the data processing system 140 is configured to analyze the collected information, optionally along with peripheral data and/or patient data, resulting in determination of one or more of a risk scale profile, an assessment recommendation to a clinician, a treatment recommendation to a clinician or a patient, and/or executable machine instructions provided directly to medical equipment such as a dialysis machine. In some embodiments, while analyzing such data in order to determine any of the above, a determination that a health event exists may occur, which may result in transmission of an alert 150 to the patient or an individual involved with the care of the patient.

As is shown, the data processing system 140 may exchange information with a patient portal 180, a clinician portal 181, one or more peripheral devices 185, and one or more dialysis machines 186. For instance, patient data such as gender, weight, age, geographic information, medication information, etc., may be obtained via the patient portal 180. In some embodiments, diagnostic data and, optionally, patient data may be obtained by the clinician portal 181. For example, the patient and/or diagnostic data may be automatically retrieved from an electronic medical record (EMR). In some embodiments, portions of either of the patient and/or diagnostic data may be obtained via user input into either of patient portal 180 or the clinician portal 181, each of which may comprise a graphical user interface (GUI) that may be accessible via a network such as the internet using a software application such as a web browser and/or an application that is configured for a particular operating system and a particular network device (e.g., Apple, Inc.'s IOS® for IPHONE®). In yet other embodiments, the patient data may be automatically obtained through web scraping, e.g., of one of a patient's social media accounts such as FACEBOOK®, INSTAGRAM®, STRAVA®, MYFITNESSPAL®, etc. The web scraping may be performed by a bot (or “web crawler”), which is logic configured to perform an automated process that searches websites for predetermined aspects, such as certain text.

In some instances, the data processing system 140 is configured with API's that enable information exchange between the data processing system 140 and the patient portal 180, the clinician portal 181, peripheral devices 185, and dialysis machines 186.

In some embodiments, the wearable biosensing device 100 may be configured to transmit collected information to a local hub 130. The “local hub” 130 constitutes logic (e.g., a device, an application, etc.) that converts a first data representation 160 of the collected information provided in accordance with a first transmission protocol (e.g., BLUETOOTH™ or other short distance (wireless) transmission protocol) into a second data representation 165. The second data representation 165 may be provided in accordance with a second transmission protocol (e.g., cellular, WiFi™, or other long distance (wireless) transmission protocol) and routes the second data representation 165 to the data processing system 140. The data processing system 140 may include an alert system (not shown), which generates and sends the alert (notification) 150 upon detecting an occurring (or potential) health event that requires attention by a clinician and/or another specified person (including the patient) responsible for addressing any occurring (or potential) health event. The alert 150 may be sent via network 170 for notification over a monitored website or application (patient or clinician portals 180, 181) or may be sent from the sensor data processing system 140 as an electronic mail (e-mail) message, a text message, or any other signaling mechanism.

Referring briefly to FIG. 1B, a block diagram illustrating a second system architecture of a wearable biosensing device coupled to set of components operating within a networked environment is shown in accordance with some embodiments. FIG. 1B illustrates many of the same components as FIG. 1A; thus, those components labeled with the same references perform the same functions and are configured in the same manner as discussed with respect to FIG. 1A unless otherwise noted.

A first distinction between the configuration of FIGS. 1A and 1B is that the data processing system 140 is located within a local hub 130 in FIG. 1B as opposed to in the network 170, e.g., on cloud-computing resources. Thus, the configuration of FIG. 1B illustrates that the data processing system 140 is stored on non-transitory, computer-readable medium of the local hub 130. The data processing system 140 as illustrated in FIG. 1B is configured to receive the same peripheral device data, patient data, biosensing data, and/or diagnostic data as in FIG. 1A. The data processing system 140 obtains such data either through push or pull transmissions that may be transmitted via the network 170. A second distinction between FIGS. 1A and 1B is that the wearable biosensing device 100 is illustrated as being disposed on a homogenously perfused tissue site (e.g., the calf muscle) of a patient instead of over the vessel 120 as shown in FIG. 1A.

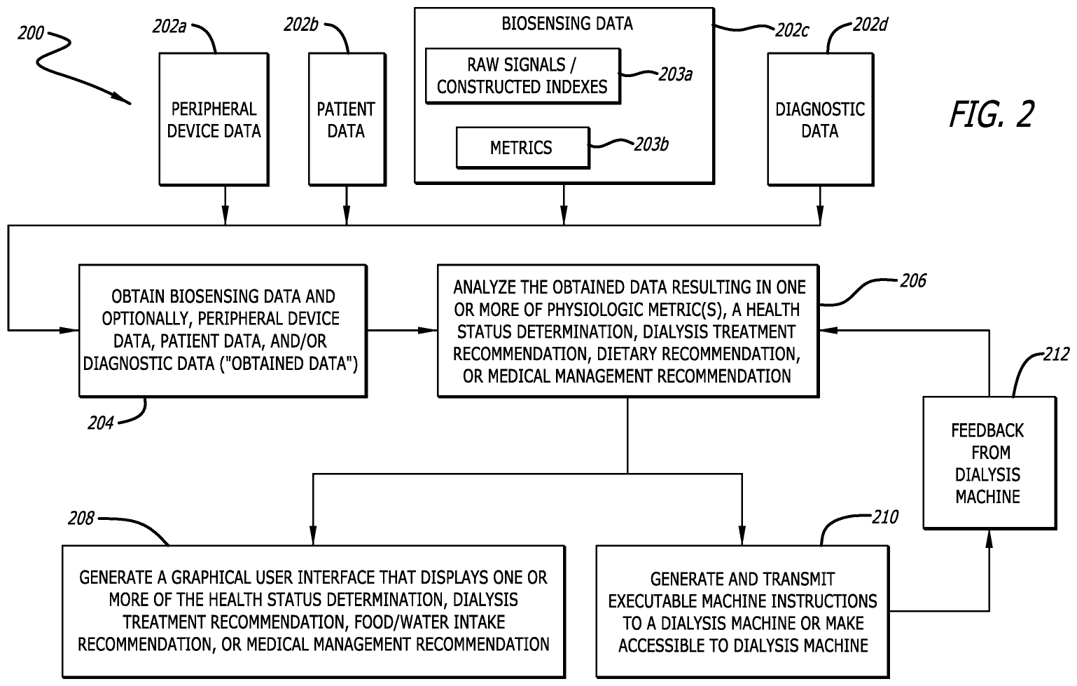

Referring now to FIG. 2, a flow diagram illustrating interoperability of the components of FIGS. 1A-1B is shown in accordance with some embodiments. Each block illustrated in FIG. 2 represents an operation performed in the method 200. It should be understood that not every operation illustrated in FIG. 2 is required. In fact, certain operations may be optional to complete aspects of the method 200. The discussion of the operations of method 400 may be done so with reference to the components of FIGS. 1A-1B.

The method 200 begins with the data processing system 140 of obtaining information such as peripheral device data 202a, patient data 202b, biosensing data 202c, or diagnostic data 202d (collectively, “obtained data 202”) (block 204). Biosensing data 202c may be comprised of raw signals/constructed indexes 203a and metrics 203b, which may refer to results of pre-processing of the raw signals/constructed indexes 203a. In some embodiments, an example of raw signals may include photoplethysmographic (PPG) and/or speckle plethysmographic (SPG) waveforms and an example of pre-processing may include determination of particular metrics from the PPG or SPG waveforms such as a blood oxygen level (e.g., the percentage of oxygen within an individual's blood, “SpO₂”) or a Hematocrit level (e.g., the percentage by volume of red blood cells within an individual's blood).

Other examples of raw signals may include data obtained from an accelerometer, a force-based sensor, an acoustic sensor, or an electrophysical sensor. An accelerometer may provide values corresponding to gravitational acceleration where a value of 1.0 represents an acceleration of 9.8 meters per second in a particular direction. A force-based sensor (also known as a force sensor or a force transducer) may refer to a transducer that is configured to measure an input applied thereto such as a mechanical load, weight, tension, compression or other pressure by converting the input to an electrical signal. An acoustic sensor may refer to a microphone configured to detect and convert sound waves to electrical signals. The acoustic sensor may be utilized to detect sound waves generated from blood passing through a vessel or near a homogenously perfused tissue site. In some embodiments, the acoustic sensor comprises a micro-electro-mechanical systems (MEMS) microphone, which is a small, silicon-based device. Examples of electrophysical sensors include electroencephalogram (EEG) and electrocardiogram (ECG) sensors. EEG and ECG sensors may comprise electrodes that measure electrical signals and resulting raw metrics may include waveforms of the measured electrical signals. The pre-processing may be performed by sensory data processing system 140a of the wearable biosensing device 110, which may represent at least a portion of the logic comprising the data processing system 140. In some embodiments, any of the obtained data 202 may be anonymized.

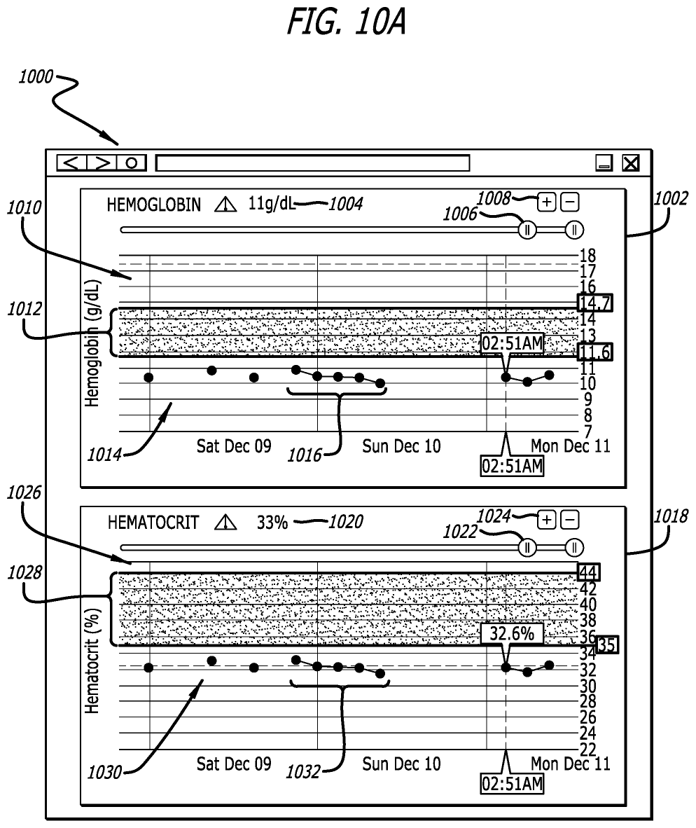

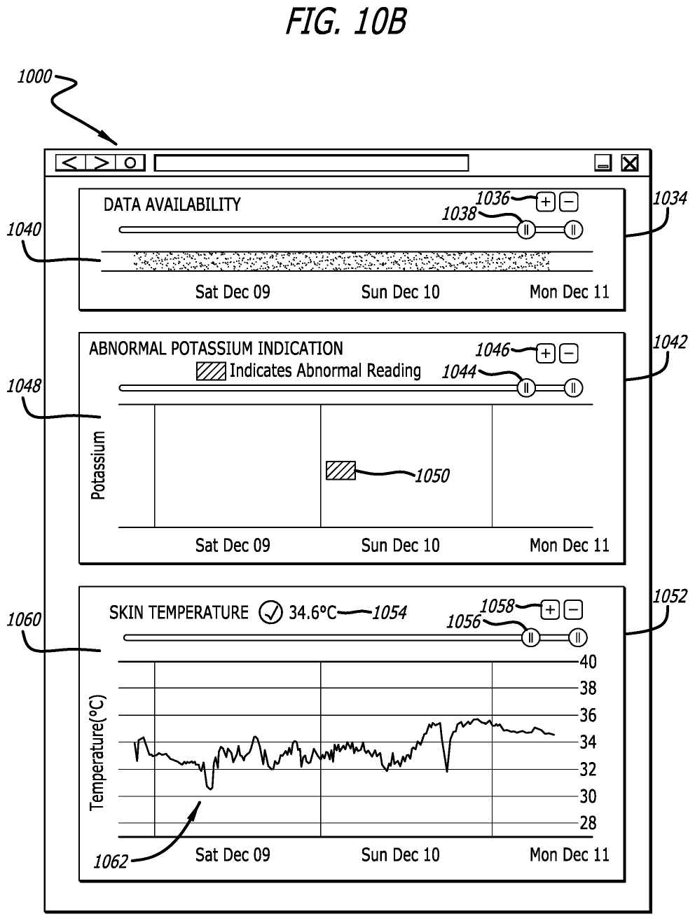

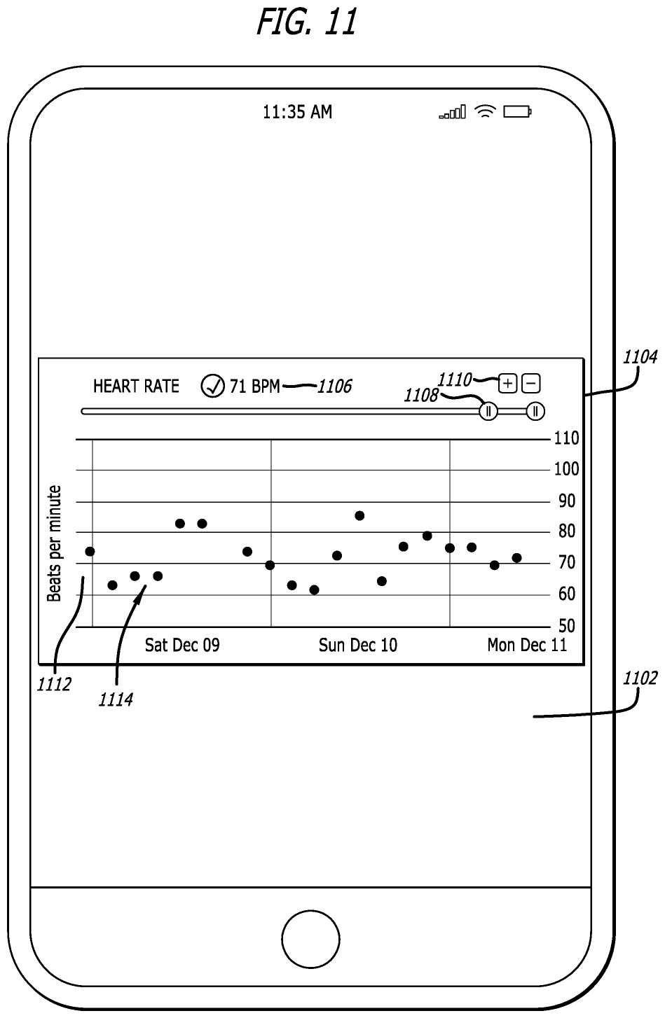

The data processing system 140 analyzes the obtained data resulting in the determination of one or more physiologic metrics, a health status determination, a dialysis treatment recommendation, a dietary recommendation, and/or a medical management recommendation (block 206). Following the determination for block 206, the method 200 may include the generation of a GUI that displays one or more of the physiologic metrics, a health status determination, a dialysis treatment recommendation, a dietary recommendation, and/or a medical management recommendation (block 208). For example, such a GUI may be displayed by the patient portal 180 and/or the clinician portal 181 of FIGS. 1A-1B. Examples of exemplary GUIs may be seen in FIGS. 10A-11.

Additionally, the method 200 may include the generation and transmission of executable machine instructions to a dialysis machine or make the instructions accessible to the dialysis machine (block 210). In some examples, the executable instructions may be transmitted to one or more dialysis machines 186 via the network 170 according to an API specifically configured to enable a dialysis machine 186 to receive such instructions, where the instructions may indicate details of a dialysis treatment for a specific patient, namely the patient 110 wearing the wearable biosensing device 100, from which the biosensing data 202c was obtained. Additionally, following the dialysis treatment indicated by the instructions, the data processing system 140 may receive feedback from the dialysis machine and/or diagnostic data such as bloodwork results (block 212). As will be discussed in detail below, the feedback from the dialysis machine and/or diagnostic data may be utilized in improving one or more machine learning models deployed by the data processing system 140. Improving the machine learning models may involve retraining of the models or otherwise correcting for bias and sensitivity, and in certain instances, retraining a model may include tailoring the model to a particular patient 110.

### III. General Device Architecture

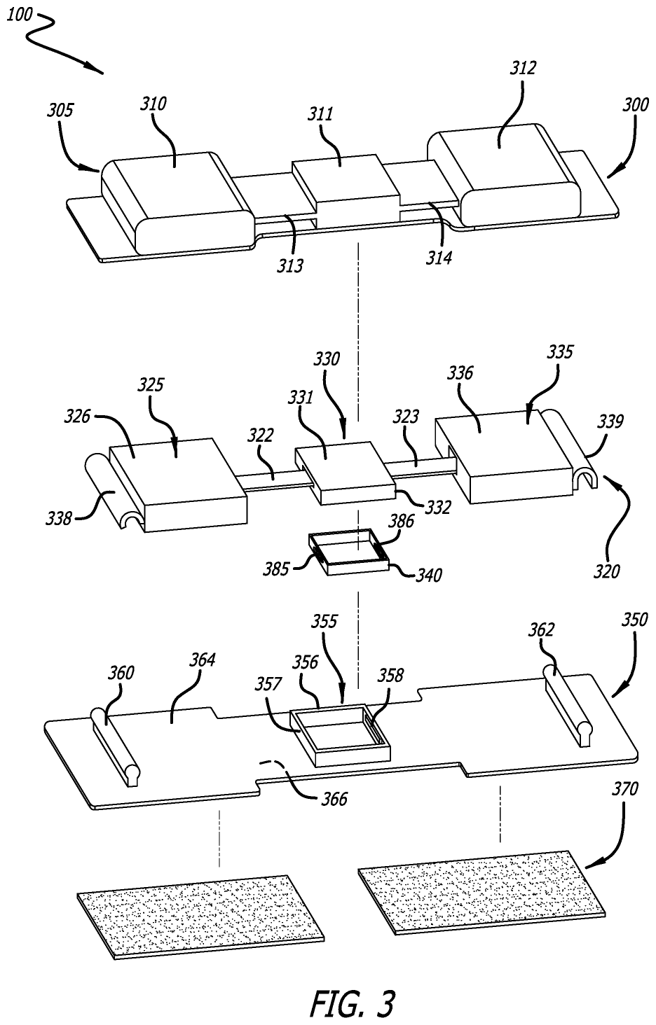

Referring to FIG. 3, an exploded view of an exemplary embodiment of the wearable biosensing device 100, which includes a first housing, a shielding component positioned under biosensing logic, a second housing, and an optional or removable adhesive layer is shown in accordance with some embodiments. For this embodiment, the wearable biosensing device 100 includes a first (top) housing 300, packaged biosensing logic 320, a shielding component 340, a second (bottom) housing 350, and an adhesive layer 370. The first housing 300 features multiple (e.g., two or more) lobes 305 formed as part of the first housing 300. The first housing 300 may be made of a flexible, water-impervious material (e.g., a polymer such as silicone, plastic, etc.) through a molding process, where the lobes 305 provide internal chambers for housing the biosensing logic 320.

As an illustrative example, according to one embodiment of the disclosure, the lobes 305 are positioned in a linear orientation, with a first plurality of lobes (e.g., first, second and third lobes 310-312) interconnected by a second plurality of lobes (e.g., fourth and fifth lobes 313-314). The fourth and fifth lobes 313-314 are configured to house interconnects 322 and 323, which provide electrical connections between an electronics assembly 325, a sensing assembly 330, and a power assembly 335 of the biosensing logic 320. As shown, each of the assemblies, namely the electronic assembly 325, the sensing assembly 330, and the power assembly 335, are maintained within a protective package 326, 331 and 336, respectively.

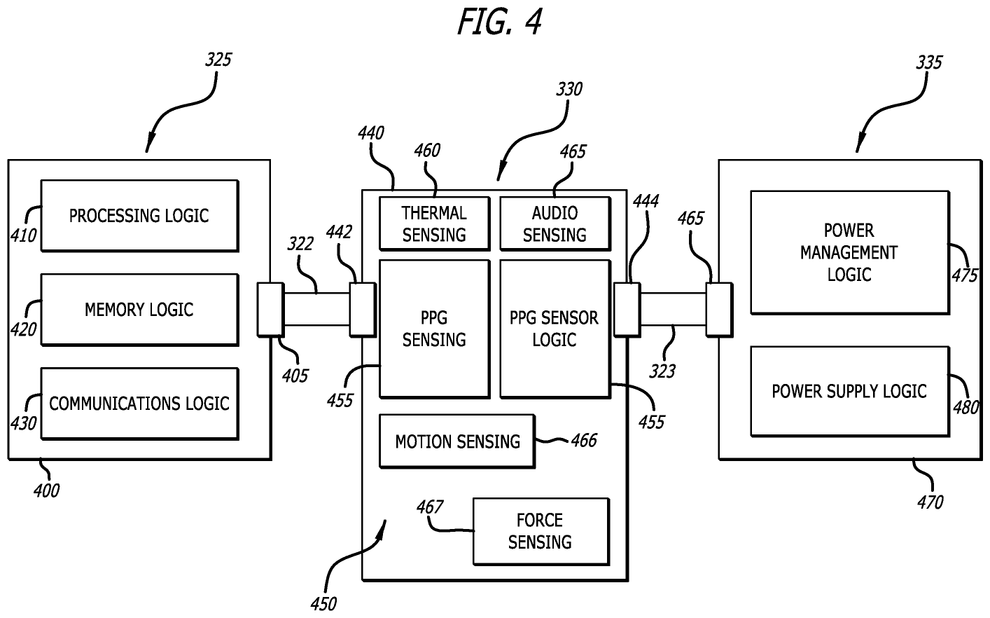

According to one embodiment of the disclosure, housed within the first lobe 310 as shown in FIGS. 3-4, the electronics assembly 325 includes a substrate 400, processing logic 410, memory logic and associated non-transitory, computer-readable medium (collectively “memory logic”) 420, and communications logic 430. Collectively, logic of the electronics assembly 325 is configured to (i) conduct analytics on data gathered by the sensing assembly 330, (ii) store the data (raw) and/or analytic results from the data, and (iii) communicate, via a wireless or a wired connection, the raw and/or the analytic results to a device (e.g., local hub 130) remotely located from the wearable biosensing device 100. For example, the electronics assembly 325 may be adapted to transit the first data representation 160 of the collected information to the hub device 130 as shown in FIGS. 1A-1B.

As further shown in FIGS. 3-4, the sensing assembly 330 is housed within the second lobe 311 of the first housing 300. The sensing assembly 330 includes a substrate 440 and one or more sensors 450 (hereinafter, “sensor(s)”) mounted on a posterior surface 445 of the substrate 440. The sensor(s) 450 may include one or more optical sensors configured to emit light via one or more light sources and/or detect reflected or refracted light via one or more light detectors. The optical sensors 450 may include a plurality of photo-plethysmograph (PPG) sensors 455 (e.g. included within PPG sensing), where each of the plurality of PPG sensors 455 includes multiple light sourcing elements and multiple light detecting elements as illustrated and described in U.S. patent application Ser. No. 18/453,194, titled “Wearable Biosensing Device with Shielding Component,” and filed on Aug. 21, 2023, the entire contents of which are incorporated herein by reference.

Referring still to FIGS. 3-4, coupled to the electronics assembly 325 and the power assembly 335 via interconnects 322 and 323, the sensing assembly 330 may be mounted on or positioned proximate to the vessel 120 of FIG. 1A or a homogenously perfused tissue site as shown in FIG. 1B. As a result, the optical sensors 450 may be used to obtain different measurements of properties of the vessel 120 or biological fluid characteristics within the vessel 120 and provide this data to the electronics assembly 325 for analysis. The optical sensors 450 may be arranged in a linear arrangement (as shown) or a circular arrangement with a light sourcing member being positioned centrally and light detecting members distributed radially from the central light sourcing member. Besides the optical sensors 450, the sensing assembly 330 may be configured to include a thermal sensing component 460 (e.g., temperature sensor, etc.), an audio sensing component 465 (e.g., microphone, etc.), a motion sensing component 466 (e.g., an accelerometer), and one or more force sensing components 467 (e.g., force transducers). The optical sensors 450 are positioned to emit or detect light via the shielding component 340 as described below.

The power assembly 335 includes a substrate 470, power management logic 475, and power supply logic 480. The power supply logic 480 is configured to provide power to both the components within the sensing assembly 330 as well as the electronics assembly 325. The power management logic 475 is configured to control the distribution of power (e.g., amount, intermittent release, or duration), including disabling of power when the wearable biosensing device 100 is not installed or detached to the wearer to avoid false data collection. The substrate 440 of the sensing assembly 330 may include hardwired traces (power layers) for routing of power from the power supply assembly 335 to components of the sensing assembly 330 and/or components of the electronics assembly 325.

Referring back to FIG. 3, the second housing 350 is configured with a centralized, raised opening 355 that is sized to surround a perimeter of the shielding component 340. Herein, according to one embodiment of the disclosure, a top surface 356 of the raised opening 355 is positioned adjacent to a bottom surface 332 of the protective package 331 for the sensing assembly 330. The raised opening 355 may further include lateral flanges 357-358, which are sized to reside within complementary lateral recesses 585-586 within the shielding component 340, which is also illustrated and described in U.S. patent application Ser. No. 18/453,194, the entire contents of which have been incorporated by reference above.

As an optional feature, the second housing 350 may include a first fastening element 360 and a second fastening element 362. These fastening elements 360 and 362 are formed on a top (anterior-facing) side 364 of the second housing 350 for attachments to complementary elements 338 and 339 positioned on outer edges of the protective packages 326 and 336, respectively. In some embodiments, such as that shown in FIG. 3, the fastening elements 360 and 362 may be inserted into and secured by fastening elements 338 and 339, and upon applying sufficient forces, the fastening elements 360 and 362 may be removed from the fastening elements 338 and 339. As a result, the first housing 300 and the second housing 350 substantially encapsulate the protective packages 326 and 336 while providing partial encapsulation of the protective package 331 inclusive of the sensing assembly 330. In some embodiments, the fastening elements 360 and 362 couple with the fastening elements 338 and 339, respectively through force-fit coupling such as where the outer diameter of the fastening elements 360 and 362 is slightly larger than the inner diameter of the fastening elements 338 and 339 and the use of force creates an interference fit. In other embodiments, the fastening elements 360 and 362 and the fastening elements 338 and 339 may be magnetic components enabling a magnetic coupling.

Additionally, the optional adhesive layer 370 is applied to at least a portion of a bottom surface 366 of the second housing 350. The optional adhesive layer 370 is adapted to attach to a surface of a patient's skin and remain attached thereto.

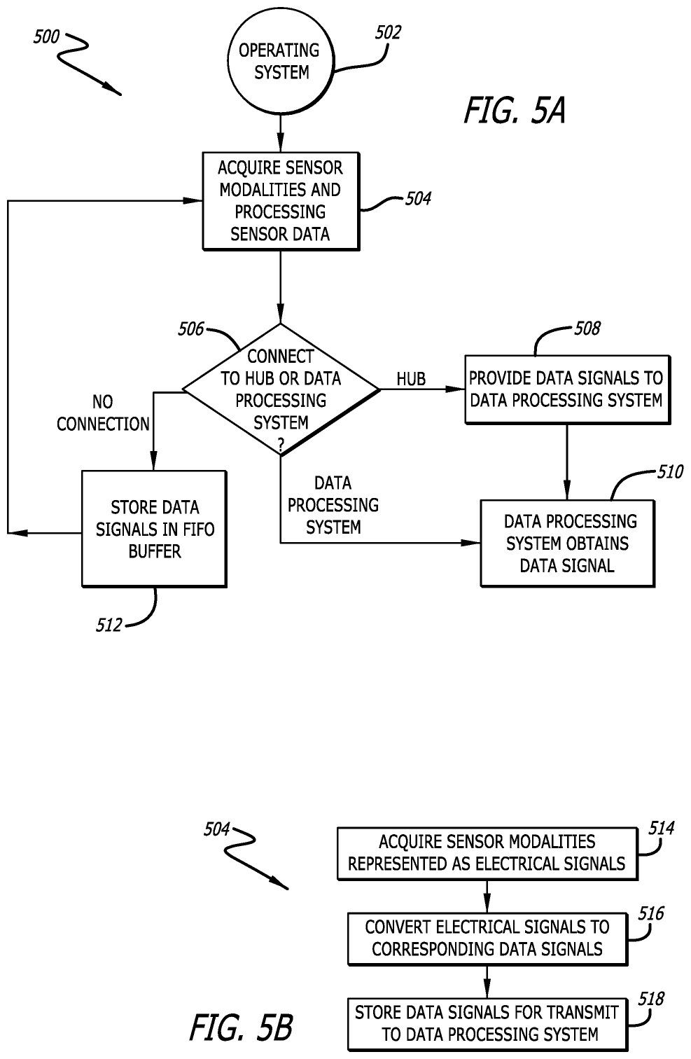

Referring to FIG. 5A, a flow diagram illustrating operations of an example operating system for the wearable biosensing device of FIG. 5A is shown in accordance with some embodiments. Each block illustrated in FIG. 5A represents an operation performed in the method 500. It should be understood that not every operation illustrated in FIG. 5A is required. In fact, certain operations may be optional to complete aspects of the method 500. The discussion of the operations of method 500 may be done so with reference to the components of FIGS. 1A-1B.

The method 500 begins as an operating system of the wearable biosensing device begins to acquire sensor modalities and process the sensor data obtained from the sensor modalities (block 502). Operations of an example method for acquiring sensor modalities and processing the sensor data are illustrated in the flow diagram of FIG. 5B. The operations of block 502 may be performed in parallel with other operations that may be performed by the operating system of the wearable biosensing device.

The wearable biosensing device attempts to establish a communicative coupling to a local hub 130 and/or the data processing system 140 (block 506). In some embodiments, the communicative coupling to the local hub 130 may include a wireless coupling via, for example, a BLUETOOTH® protocol or other wireless protocols. In other embodiments, a communicative coupling may be established with the data processing system 140 through a network connection. In such embodiments, credential data or other authorization or identification data may be exchanged between the data processing system 140 and the wearable biosensing device 100 establishing a communicative coupling, where such data may be transmitted via a network, which may refer to one or more local area networks (LANs), wide area networks (WANs), cellular networks (e.g., long term evolution (LTE), High Speed Download/Upload Packet Access (HSDPA, HSUPA, HSPA, HSPA+), 3G, and other cellular technologies), and/or networks using any of terrestrial microwave, or satellite links, and may include the public internet.

When the wearable biosensing device communicatively couples with the hub 130, biosensing data may be transmitted to the hub 130 via either a push or pull transmission (block 508). For instance, the biosensing device may actively transmit biosensing data (push). Alternatively, the hub 130 may retrieve the biosensing data (pull). In either embodiment, specific APIs may be utilized to ensure the transmission is obtained by the hub 130 in a format that is readable or usable by the hub 130. In some embodiments, the hub 130 performs any of a portion of processing operations applicable to the biosensing data disclosed herein prior to transmitting such to the data processing system 140. In embodiments, the hub 130 may transmit the biosensing data along with any processed data to the data processing system 140. In other embodiments, the hub 130 merely operates as a relay to pass the biosensing data obtained from the wearable biosensing device to the data processing system 140.

When the wearable biosensing device communicatively couples with the data processing system 140 (i.e., without use of the hub 130), biosensing data may be transmitted to the data processing system 140 via either a push or pull transmission in the same manner as discussed above (block 510).

However, when a communicative coupling cannot be established with either the hub 130 or the data processing system 140 (no at block 506), the operating system of the wearable biosensing device stores the biosensing data (data signals) in a buffer in memory of the biosensing device such as in the memory logic 420 as illustrated in FIG. 4. In some embodiments, the non-transitory, computer-readable medium of the memory logic 420 may include a First-In-First-Out (FIFO) buffer (block 512). Once the data signals are written to the FIFO buffer, the method 500 may return to acquiring sensor modalities and processing sensor data (block 504).

In some embodiments, storing of the data signals in the FIFO buffer may occur following expiration of a connection timer that is initiated upon attempting to connect to either the hub 130 or the data processing system 140. When the connection timer reads timeout, the wearable biosensing device may write the data signals to the FIFO buffer and delete the oldest data signals as needed. Additional details and embodiments pertaining to connecting the biosensing device to a hub device or data processing system are discussed in more detail in U.S. Pat. No. 11,406,274, titled “Wearable Device with Multimodal Diagnostics,” which issued on Aug. 9, 2022, the entire contents of which are incorporated herein by reference.

Referring to FIG. 5B, a flow diagram illustrating detailed operations comprising the acquisition of sensor modalities and processing sensor data is shown in accordance with some embodiments. Each block illustrated in FIG. 5B represents an operation performed in the block 504 of FIG. 5A. It should be understood that not every operation illustrated in FIG. 5B is required. In fact, certain operations may be optional. The discussion of the operations in FIG. 5B may be done so with reference to the components of FIGS. 1 and 5A.

The method 504 begins as the wearable biosensing device begins to acquire sensor modalities, which may be represented as light and/or electrical signals (block 516). The light and/or electrical signals may be converted to sensor data signals (block 518). The conversion may be performed by a processor of the wearable biosensing device using, for example, an analog-to-digital (ADC) function. For example, the processor may receive analog audio input signals from the audio sensing component 465 (e.g., microphone) of FIG. 4 and convert the analog signals to digital samples. The conversion of electrical signals to sensor data signals may also be performed at least partially by the sensor component. For example, a sensor such as the accelerometer, temperature sensor, or any other modular sensor component, for example, may be provided with ADC functions as well as an appropriate bus interface that permits digital communication with the processor. The processor may then perform any signal conditioning functions, such as filtering or other signal processing functions before storing the sensor data signals for later transmission to the hub 130 and/or the data processing system 140 (block 520).

### IV. Logical Architecture and Operability Thereof

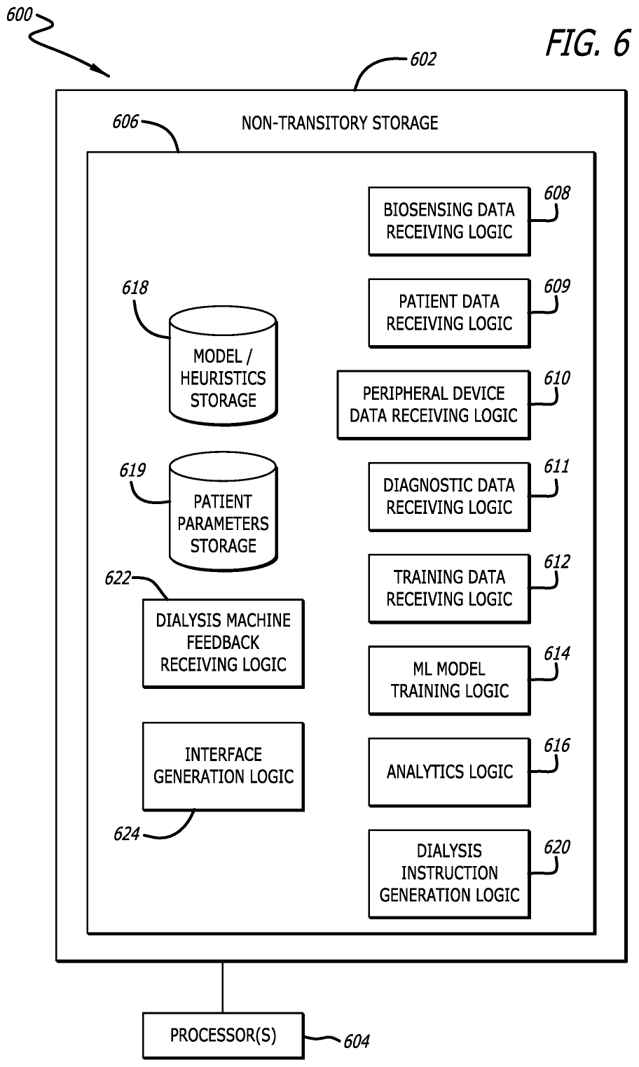

Referring to FIG. 6, a logic diagram illustrating a logic modules of a diagnostic and treatment determination system (“system”) 606 stored on non-transitory storage 602 and configured to be executed by one or more processors 604 is shown in accordance with some embodiments. The computing environment 600 shown to include the non-transitory storage 602 and the processors 604 may represent a portion of a network device, e.g., a server device, a mobile phone, a tablet, or other computing device. In other embodiments, the computing environment 600 may represent a cloud-computing environment, which may be understood to comprise hosted services delivered via a network, such as the internet. In some instances, the computing environment 600 may refer to cloud computing services including servers, storage, and virtualized compute resources hosted by a third-party, such as Amazon Web Services, Inc. (AWS), where the system 606 is configured for processing within a virtualized computing environment, e.g., a virtual machine (VM).

The system 606 includes logic, which may comprise one or more logic modules formed of executable instructions specifically configured to cause performance of operations when executed by the processors 604. The system 600 is shown to include a biosensing data receiving logic 608, a patient data receiving logic 609, a peripheral device data receiving logic 610, a diagnostic data receiving logic 611, a training data receiving logic 612, a machine learning (ML) model training logic 614, an analytics logic 616, a dialysis instruction generation logic 620, a dialysis machine feedback receiving logic 622, and an interface generation logic 624. Additionally, the system 600 may comprise various storage modules, which may of course be combine into one or more modules. These storage modules may include a model/heuristics storage (“model storage”) 618, and a patient parameters storage 619.

Each of the biosensing data receiving logic 608, the patient data receiving logic 609, the peripheral device data receiving logic 610, the diagnostic data receiving logic 611, and the training data receiving logic 612 may each be configured, upon execution by the processors 604, to receive or otherwise obtain specific data from one or more particular devices or as user input. The biosensing data receiving logic 608 is configured to obtain biosensing data from the wearable biosensing device 100, which may be performed through a communicative coupling utilizing a set of APIs specific to the wearable biosensing device 100. In some instances, the wearable biosensing device 100 may be configured to communicatively couple with a software or firmware application processing on a separate network device in a similar manner as discussed below with respect to the peripheral device data receiving logic 610.

Similarly, the peripheral device data receiving logic 610 is configured to obtain peripheral device data from one or more peripheral devices such as a body weight scale, a heart rate monitor, a wearable fitness tracker, etc., where each peripheral device may exchange data with the peripheral device data receiving logic 610 (e.g., via a network connection). In some instances, the peripheral device data receiving logic 610 may be configured to obtain peripheral device data from a software or firmware application that processes on a separate networking device, such as a mobile phone or tablet. In one example, a body weight scale (peripheral device) may be communicatively coupled with a software application processing on a user's mobile phone such that the body weight scale transmits data (peripheral device data) to the user's mobile phone, and the software application may relay the peripheral device data from the body weight scale to the peripheral device data receiving logic 610.

The patient data receiving logic 609 and the diagnostic data receiving logic 611 may be configured to receive patient data and diagnostic data respectively, each of which may be obtained through access to an electronic medical record (EMR). For instance, the patient data receiving logic 609 and the diagnostic data receiving logic 611 may be configured to access an EMR system, such as that of a hospital, clinical practice, etc. In some instances, the receiving logics 609, 611 are configured to receive user input being credential or authorization information, which is then provided to the EMR system enabling the receiving logics 609, 611 to access the EMR corresponding to a particular patient (e.g., the wearer of the wearable biosensing device 100). In other embodiments, the receiving logics 609, 611 may be configured to receive patient data and/or diagnostic data as user input received through a graphical user interface (GUI) generated by the interface generation logic 624.

The training data receiving logic 612 is configured to obtaining training data to be utilized in training one or more machine learning models and/or neural networks that will be discussed in greater detail below. In some examples, the training data receiving logic 612 obtains training data by accessing and retrieving such from the model storage or another data store (not shown). In other examples, the training data receiving logic 612 obtains training data through user input provided to a GUI generated by the interface generation logic 624.

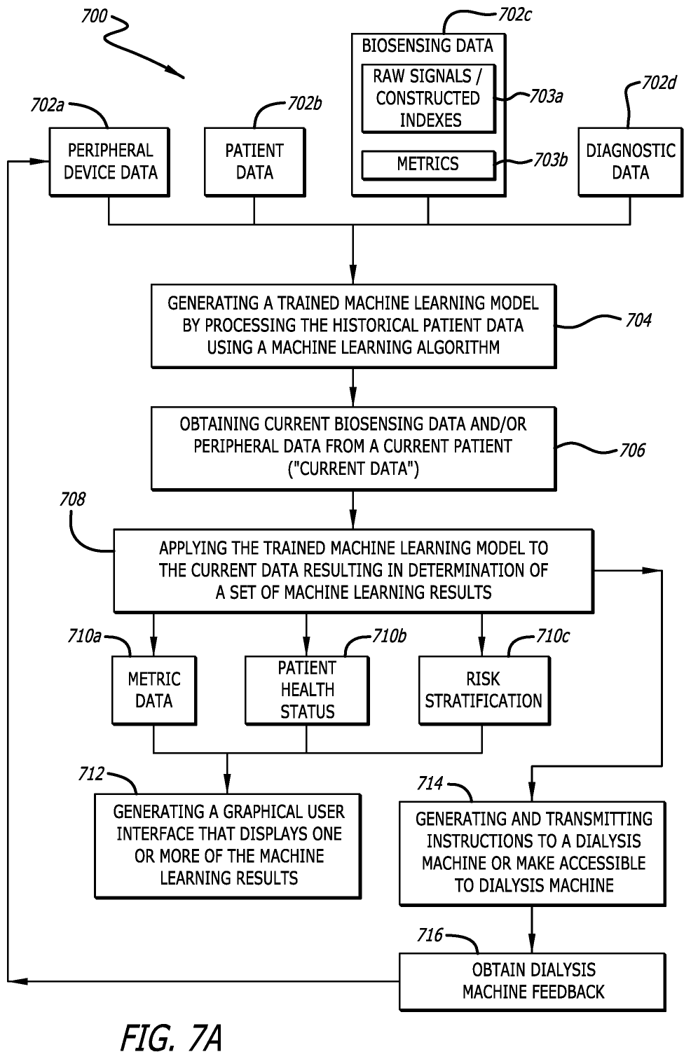

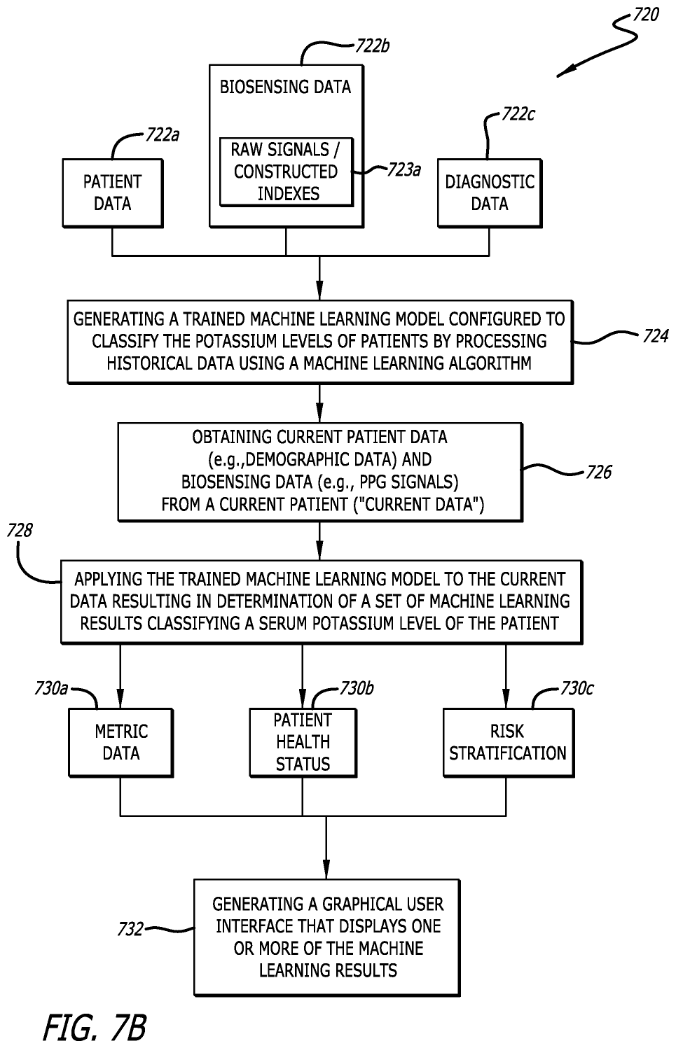

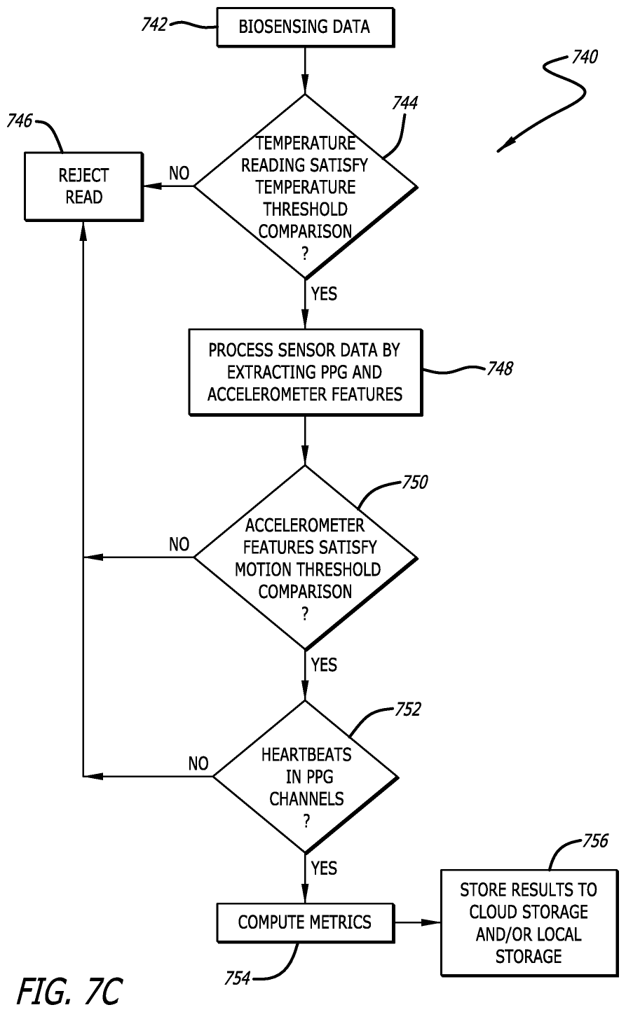

The analytics logic 616 is configured to perform various analytics on received data (e.g., biosensing data, diagnostic data, patient data, and/or peripheral data). As illustrated in further detail in FIGS. 7-9, the analytics logic 616 may perform operations categorized as one or more artificial intelligence techniques. For example, the analytics logic 616 may perform operations of generating and implementing a trained machine learning (ML) model, a neural network, or a deep learning neural network.

### 1. Machine Learning Models

More specifically, the analytics logic 616 may perform operations of generating and implementing a trained machine learning (ML) model, where generation is performed generally through processing of historical data with a ML algorithm. The trained ML model may be specifically configured to receive as input any of biosensing data, diagnostic data, patient data, and/or peripheral data (which may be dependent on the historical data utilized in training). As should be understood, machine learning is a subset of artificial intelligence (AI) that involves the development of algorithms and models that enable computers to learn and make predictions or decisions based on data, without being explicitly programmed. In essence, the goal of machine learning is to allow computers to improve their performance on a task over time by automatically learning from examples.

The implementation of machine learning by the analytics logic 616 may include the operations of data collection, data preprocessing, feature engineering, model training, loss function optimization, validation/testing, hyperparameter tuning, and deployment/inference. More specifically, data collection includes obtaining a dataset that contains examples relevant to the task to be learned by the machine learning model. The dataset includes input features (also known as attributes) and corresponding target labels (the desired output or outcome). As raw data (comprising the dataset) is often messy and may contain noise, missing values, or inconsistencies, data preprocessing operations may involve automated cleaning and transforming of raw data into a usable format. This may include signal filtering, signal conditioning, feature engineering, removing outliers, filling in missing values, and feature scaling.

Feature engineering includes determination of input features within the dataset that are specific for the task to be learned in order to represent the underlying patterns in the data effectively. Feature engineering involves the selection of the relevant features and, in some instances, the creation of new features to enhance the trained ML model's performance. Through feature engineering, the set of input features (collectively, the “input vector”) may include characteristics of one or more sensor signals obtained from the biosensing device, as well as data obtained from one or more peripheral devices, a patient's electronic health record, and/or feedback from a dialysis machine.

The target labels may be continuous or discrete in nature, where the nature of the target labels informs the selection of the ML architecture or algorithm used for the specific task. As one illustrative example, the target labels may be continuous serum potassium concentration values obtained via laboratory analysis of whole blood samples. In such an instance, a regression model may be trained and configured to provide predictions related to altering or maintain serum potassium concentration values in a patient's blood. As a second illustrative example, the target labels may be a stratified determination of serum potassium “status” based upon thresholds, which may be commonly accepted in the medical community. In such an example, a classification model may be trained and configured to provide a prediction of a patient's serum potassium status.

In either illustrative example, e.g., training and deploying either a regression or classification model, the input vector may include features from optical sensor signals obtained using a plurality of light wavelengths (where an optical sensor signal refers to a recorded waveform itself). These optical features may include time-domain features such as the signal amplitude, period, and/or location within the period of features associated with the cardiac cycle. Additional features may include prior predictions of patient health metrics, such as blood hemoglobin, and demographic information, such as patient skin pigmentation, age, height, and weight.

The combination of features may be determined using an automated or semi-automated feature selection procedure. Features may be removed if they provide little information or if they are redundant with other features. In the latter case, pseudo-redundant features may be combined to create a more robust single feature; for example, the median feature value from two or more optical channels using the same light wavelength may be used in place of the individual feature values from the two or more optical sensor signals. In addition to feature selection, model hyperparameters may be tuned to optimize the model's predictive performance. Hyperparameters may refer to model configurations that are not learned during training but affect the model's behavior.

For ensemble learning models such as a random forest, these hyperparameters may include the number of weak learners that comprise the ensemble learner, the size of those learners, and the maximum number of features that can be used to make a prediction. If a boosting algorithm is being used, the learning rate, or weight placed on each additional “boosting” learner, may also be tuned.

Model training includes providing a selected ML algorithm with the prepared dataset (e.g., following preprocessing and including the selected features) such that processing of the prepared dataset by the ML algorithm results in a trained model through adjustment of a model's internal parameters causing a mapping of the input features to the target labels. The ML algorithm utilized for training may be dependent on the task to be solved. As noted above, the analytics logic 616 may include one or more trained ML models that are configured to determine one or more of a risk score determination, an assessment recommendation, a treatment recommendation for a clinician or patient, and/or executable dialysis machine instructions. Example ML algorithms may include tree-based ensemble learning models (such as extreme gradient boosting (XGBoost) and random forest), clustering algorithms (such as k-nearest neighbors and support vector machines), and/or neural networks with architectures that may include artificial neural networks, residual neural networks, etc. The example ML algorithms may be utilized in training a ML classification model that is configured to determine prediction of a health stratification or discrete risk score. Additionally, the example ML algorithms may be utilized in training a ML regression model that is configured to determine a quantitative prediction of a continuous health metric such as blood hemoglobin. In some instances, the trained ML model is configured to provide a combination of the prediction of a health stratification or discrete risk score and a continuous health metric.

During the model training process, operations are performed to minimize a loss function (also known as a cost function), which quantifies the difference between the model's predictions and the actual target labels. The loss function depends on the ML algorithm utilized in training; for example, mean squared error may be used for regression tasks and cross-entropy may be used for classification tasks. Additional details of minimizing the loss function are described below but as a general summary, the model's internal parameters are updated iteratively using techniques such as gradient descent, which is a first-order iterative optimization algorithm for finding a local minimum of a differentiable function such that the parameters of the model are adjusted in the direction that reduces the loss function.

Following training, the model's performance is evaluated on data not utilized in training (e.g., data not previously presented to the model). The dataset is usually split into a training set (used for training) and a validation/test set (used for evaluation). The model is provided the validation/test set as input (without labeling) and the resulting prediction/determination is evaluated against the labeling of the validation/test set. Depending on the machine learning algorithms utilized, hyperparameters of the model may be tuned following training, or iteratively as part of the training process.

Following validation and testing of the model, the model may be deployed to make predictions on new, real-world data (e.g., patient data, peripheral device data, biosensing data, diagnostic data, etc.). During inference, the model processes new input data and generates predictions or decisions based on what it has learned during training.

Although the following subsections provide detail as to training and deployment of a linear regression model and neural network, the disclosure is not limited to these examples. The following is merely intending to be illustrative as to possible artificial intelligence techniques. Additionally, it should be understood that either a linear regression model or a neural network may be used to determine a risk score, an assessment recommendation, and/or a treatment recommendation to be provided to a clinician and/or a patient. In some instances, the treatment recommendation may include parameters for a dialysis treatment such as the blood flowrate, dialysate flow rate, and/or the duration of a dialysis treatment. In some instances, the dialysis treatment parameters may be provided to the dialysis instruction generation logic 620 as discussed below.

### A. Linear Regression

As one example, the implementation of machine learning by the analytics logic 616 may include training and deployment of a linear regression machine learning model. Training of a linear regression machine learning model involves finding the best-fitting linear relationship between a set of input features and a target variable. This relationship is represented by the known linear equation of:

$$y = {β}_{0} + {β}_{1 {x}_{1}} + {β}_{2 {x}_{2}} + … + {β}_{n {x}_{n}}$$

Where y is the target variable to predict, β₀ is the intercept (y-axis value when all x values are 0), and β₁, β₂, . . . , βₙ are the coefficients for the respective input features. Training a linear regression model results in determining the values of β₀, β₁, α₂, . . . , βₙ that minimize the error between the predicted values (y) and the actual target values. This is typically done using a mathematical optimization technique known as least squares regression. In some embodiments, the operations involved in training a linear regression model include data collection, data preprocessing, model initialization, coefficient minimization, and model evaluation. In other instances, maximum likelihood estimation (MILE) may be utilized in determining the parameters for the linear regression model. With respect to a linear regression model, MILE maximizes the likelihood of observing the given data under the assumed distribution of errors (e.g., normal distribution), which results in a determination of parameter values that make the observed data (such as the training data) most probable. Differently, least squares regression minimizes the sum of the squared differences between the observed and predicted values with the objective being to minimize the sum of the squared residuals (vertical distances between data points and the regression line).

A dataset (training data) is obtained that includes input features (x₁, x₂, . . . , xₙ) and the target variable (y). The training data may undergo preprocessing steps including missing value handling, scaling, or normalizing the values. The coefficients (β₀, β₁, β₂, . . . , βₙ) are initialized such as with a value of 0 or a small random values. Next, the values of the coefficients are determined that minimize a loss function, e.g., the mean squared error (MSE), which is typically performed through the use of the gradient descent algorithm. This is an iterative process including predicting a first value for y (y_{predict}) using the initial (current) coefficient values and input features. The error (or loss) between y_{predict} and the target value of y. The coefficient values are then adjusted using an optimization algorithm (e.g., gradient descent). The process iterates until the error or loss either no longer improves or no longer improves above a threshold amount, or a predetermined number of iterations have been performed. Following validation and testing of the model, the model may be deployed to make predictions on new, real-world data (e.g., patient data, peripheral device data, biosensing data, diagnostic data, etc.).

b. Neural Networks

Additionally, or alternatively, the analytics logic 616 may perform operations to train and implement a neural network configured to determine one or more of a risk score determine, an assessment recommendation, a treatment recommendation for a clinician or patient, and/or executable dialysis machine instructions. As should be understood, a neural network consists of layers of interconnected nodes, or “neurons,” organized into three main types of layers: input layer, hidden layers, and output layer. Each neuron in a layer is connected to neurons in the adjacent layers through weighted connections.

More specifically, the input layer receives the raw data or features (input data) relevant to the task assigned to the neural network, wherein each neuron in the input layer corresponds to a specific feature in the input data. The hidden layer(s) are intermediate layers between the input and output layers that perform complex transformations and feature extraction. Each neuron in a hidden layer takes inputs from the neurons in the previous layer, applies weights to those inputs, and passes the result through an activation function. Each connection between neurons has an associated weight that determines the strength of the connection and affects how information is propagated through the layers of the neural network. Neurons in a hidden layer apply an activation function to the weighted sum of their inputs. The activation function introduces non-linearity to the network resulting in detection of complex relationships within the input data. Examples of activation functions include ReLU (Rectified Linear Activation), sigmoid, and tanh. The output layer produces the final predictions or decisions of the neural network. The number of neurons in this layer depends on the task the neural network is assigned to solve. For example, a binary classification problem may include a single neuron with a sigmoid activation function, while a multi-class classification problem may include multiple neurons with softmax activation.

The training of a neural network involves multiple steps including a feedforward pass, computing a loss function, backpropagation, and optimization (updating weights). More specifically, the process of feeding data through the network, computing loss, performing backpropagation, and updating weights is repeated iteratively for multiple epochs (passes through the entire training dataset) until the neural network's performance converges to a satisfactory level in a similar manner as discussed above with respect to training a machine learning model.

In explaining the training process in more detail, during a feedforward pass, input (training) data is fed into the neural network. The data passes through the layers, and each neuron's weighted inputs are transformed using the activation function. The output of the output layer represents the neural network's prediction. Subsequently, the neural network's predictions are compared to the actual target values (which are known as part of the training data) using a loss function. The loss function quantifies how well the neural network's predictions match the desired outcomes. Examples of loss functions include mean squared error for regression and cross-entropy for classification. Once the loss is computed, the neural network's parameters (weights and biases) are adjusted to minimize the loss. Backpropagation is the process of computing the gradients of the loss with respect to the weights and biases, where the gradients indicate the direction and magnitude of changes needed to minimize the loss. The gradients are computed layer by layer, starting from the output layer and working backward toward the input layer using an optimization algorithm (e.g., gradient descent). For example, gradient descent causes results in a determination of an adjustment to be made for each parameter of the model in the opposite direction of the gradients, effectively moving the parameters towards values that reduce the loss. The weights and biases are then updated according to the computed gradients.

As noted above, the training process is repeated iteratively for multiple epochs (passes through the entire training dataset) until the neural network's performance converges to a satisfactory level, in a similar manner as discussed above with respect to training a machine learning model.

Deployment of a trained neural network comprises a feedforward pass, where input data is fed into the trained neural network. The data passes through the layers, and each neuron's weighted inputs are transformed using the activation function. The output of the output layer represents the network's prediction.

Referring again to FIG. 6, the dialysis instruction generation logic 620 may be configured to generate instructions, which in some instances may include dialysis parameters such as the blood flowrate, dialysate flow rate, and/or the duration of a dialysis treatment. In some embodiments, the dialysis instruction generation logic 620 may receive results from the analytics logic 616 including the dialysis treatment parameters as noted above. In some instances, the dialysis treatment parameters may then be inserted into a software module template (e.g., a source code template), which may be compiled thereby converting the source code including the dialysis treatment parameters determined by artificial intelligence into executable code, configured to be executed by the processor of a dialysis machine.

The dialysis feedback receiving logic 622 may be configured to receive feedback pertaining to a timing and completion of a dialysis treatment directly from a dialysis machine. For example, the data processing system 140 may be configured to exchange information with a dialysis machine over a network via a set of APIs. The information exchanged may include executable machine instructions described above and/or feedback as to metadata of a dialysis treatment (or lack thereof). Example metadata may include, a completion status, the dialysis treatment parameters, a timing of the dialysis treatment, a location of the dialysis treatment, etc. Additionally, the dialysis feedback receiving logic 622 may be configured, separately or jointly with the diagnostic data receiving logic 611, to receive bloodwork data following a dialysis treatment. Thus, the dialysis feedback receiving logic 622 may associate treatment metadata with bloodwork from the treatment for storage together. The received feedback may be utilized in subsequent training (or “retraining”) of a machine learning model.

The interface generation logic 624 is configured to generate graphical user interfaces (GUIs) that are configured for display on one or more network devices. The physical screens of the network devices may differ in dimension such that the GUIs are generated to properly display on screens of various dimensions. Additionally, the GUIs may be generated for display by different methodologies such as via a website browser, such as the CHROME™ browser by Google, LLC, or a software that is installed on a particular network device for the dedicated purpose of enabling the wearer of a wearable biosensing device to obtain insights and/or recommendations about their health or treatments. Examples of GUI display screens that may be generated by the interface generation logic 624 are illustrated in FIGS. 10A-11.

### V. Example Methodology Flows

Referring now to FIG. 7A, a flow chart illustrating operations performed by the system of FIG. 6 to train and apply a machine learning model to generate a prediction of the health status of a patient is shown in accordance with some embodiments. Each block illustrated in FIG. 7A represents an operation performed in the method 700. It should be understood that not every operation illustrated in FIG. 7A is required. In fact, certain operations may be optional. The discussion of the operations in FIG. 7A may be done so with reference to the components illustrated in other figures or otherwise described herein.

The method 700 begins when the data processing system 140 of FIG. 1 obtains information such as peripheral device data 702a, patient data 702b, biosensing data 702c, or diagnostic data 702d (collectively, “historical data 702”). It should be understood that the obtained data illustrated in FIG. 7A refers to the same obtained data 202 illustrated in FIG. 2.

From the historical data 702, a trained machine learning model is generated by processing obtained data (e.g., historical data 702) using a machine learning algorithm (block 704). As discussed above in detail, embodiments of the disclosure may implement different artificial intelligence or machine learning techniques such as linear regression, logistic regression, neural networks, decision trees, Naïve Bayes as examples. Thus, use of the term “machine learning” at least in FIG. 7A broadly refers to techniques within the field of artificial intelligence, which is understood to encompass machine learning, which itself is understood to encompass deep learning, e.g., neural networks.

Following the training of a machine learning model, the method 700 continues by obtaining current biosensing data and/or peripheral data from a current patient (“current data”) (block 706). The trained machine learning model is then applied to the current data resulting in a set of machine learning results (block 708), which includes one or more of predictions of future or current metric data 710a, a current or future patient health status 710b, and/or any phase result of a risk stratification 710c (as discussed above). The set of machine learning results (710a-710c) may be provided in one or more graphical user interfaces (block 712), which may include, for example, a patient portal and/or a clinician portal. Additionally, as an optional step, instructions may be generated that are configured to be provided to and carried out by a dialysis machine (block 714). Additionally, in some embodiments, feedback may be obtained from the dialysis machine, regardless of whether instructions are generated and provided to the dialysis machine (block 716), which is fed back into the machine learning model as peripheral device data 702a. For instance, the feedback from the dialysis machine may be a component within feedback system such as a proportional, integral, and derivative (PID) control system.

### a. Example Use Case—Potassium Imbalance

In one illustrative example, the data processing system 140 deploys a machine learning classification model trained and configured based at least on biosensing data obtained through use of the wearable biosensing device to differentiate patients with hypokalemia (K+<3.5 mEq/L) or hyperkalemia (K+>5.2 mEq/L) from patients with normal potassium levels. In this example, the input data comprise features derived from optical and mechanical sensors within the wearable biosensing device, and the output predictions were ternary classification of routine blood potassium test results pre- and post-dialysis: hyperkalemia (>5.2 mEq/L), hypokalemia (<3.5 mEq/L) or normokalemia (3.5-5.2 mEq/L).

One method of obtaining training data included providing patients with a wearable biosensing device, where the biosensing device is placed over a mid-portion of a dialysis access (e.g., arteriovenous fistula or graft). Reference potassium measurements for each patient were obtained by standard laboratory analysis of two blood samples: one taken before a hemodialysis session and one after the session, where the blood draws were performed within, for example, 15 minutes of the beginning, or end of the dialysis session, with a mean time of seven minutes between the pre-dialysis blood draw and the beginning of the session and a mean time of five minutes between the end of the session and the post-dialysis session. Additionally, biosensing data was obtained within, for example, five minutes of these blood draws. The wearable biosensor data is then uploaded to a cloud-based data processing system for analysis and evaluation using an encrypted architecture.

One such wearable biosensing device includes an optical sensor module with twelve different source-detector paths and three wavelengths: red (660 nm), green (530 nm) and infrared (940 nm). Each optical channel captures a discrete photoplethysmography (PPG) waveform that is preprocessed by down-sampling and applying a finite impulse response (FIR) filter to eliminate noise and highlight relevant features. The parameters of the FIR filter are adjusted based on the detected heart rate in each data recording to provide consistent waveform features regardless of the patient's heart rate, which can vary substantially even in healthy patients. Each optical signal is analyzed to extract features that relate to the cardiac cycle, which are fed into a machine learning model trained and configured to identify excursions in serum potassium concentration by quantifying changes to patient hematocrit (Hct) levels and the systolic-diastolic periods. These results are subsequently correlated with an imbalance in serum potassium levels.

As an illustrative example, each of the PPG sensors 455 includes one or more light sourcing elements. Herein, the light sourcing elements may include light emitting diodes (LEDs) of different wavelength ranges such as one or more LEDs emitting light with wavelengths ranging between 520-540 nanometers (nm) (e.g., green LED with light emissions of approximately 532 nm), one or more LEDs emitting light with wavelengths ranging between 645-665 nm (e.g., red LED with light emissions of approximately 655 nm), and one or more LEDs emitting light with wavelengths ranging between 930-950 nm (e.g., infrared “IR” LED with light emissions of approximately 940 nm). Besides the light sourcing elements, the sensing assembly 230 further includes light detecting elements, which may include one or more photodiodes (photodetectors) configured to capture reflected or refracted light emitted from a light sourcing element after traveling across an optical path with passage to or through a vessel or homogenously perfused tissue sites.

As a second illustrative example, each of the PPG sensors 455 includes one or more light sourcing elements. Herein, the light sourcing elements may include laser diode (e.g., a 785 nm laser diode) and a detector (e.g., a 752-pixel×480-pixel CMOS array or a lenseless camera module (e.g., a global shutter)). The detector is configured to collect refracted or reflected light. In implementations in which the detector is a lenseless camera module, the collected light may be referred to as speckle data. The refracted or reflected light may be captured at 50, 100, 200, etc., frames per second, where the frames of light are processed to generate PPG or SPG waveforms, or estimates thereof.

In some instances, the machine learning model is an Extreme Gradient Boosting (XGBoost) classification model trained and configured to distinguish between patients with hypokalemia (K+<3.5 mEq/L), hyperkalemia (K+>5.2 mEq/L) and normal potassium levels. In one deployment, a combined set of 1229 data recordings were used, and parameter tuning and performance evaluation were performed using nested k-fold cross validation. A 3-fold cross-validated grid search hyperparameter optimization was performed to determine the optimal set of model parameters, which were then fed into a 10-fold cross-validated XGBoost classification model to produce classification predictions of patient potassium status. Boosted model architectures like XGBoost tend to outperform other ensemble learning methods, such as random forest.

As is understood, each cardiac cycle of the heart pumps blood to the body and is detectable as a pressure pulse. The data processing system 140 seeks to detect the changes in volume caused by the pressure pulses by illuminating the skin with light from, for example, an LED on a PPG sensor 455 and then measuring the amount of light reflected to a photodiode on the PPG sensor 455. The PPG obtained from the PPG sensor 455 includes peaks representing each cardiac cycle. Because blood flow to the skin can be modulated by multiple other physiological systems, the PPG can also be used to monitor breathing, hypovolemia, and other circulatory conditions, such as for example a level of stenosis of a vessel. In some example implementations, a motion sensor (accelerometer 466), the PPG sensor 455, and the acoustic sensor 465 can be utilized in conjunction with one another to provide monitoring of additional physiological parameters. Thus, reflected light may be converted into PPG signals such that examples of features that may be analyzed by the machine learning model include but are not limited to: amplitude (peak), height, area, width, and/or maximum and minimum slope.

Examples of metric data (e.g., 710a) predicted by the data processing system 140 may include machine volumetric blood flow rate, hematocrit, oxygen saturation, change in blood volume, and/or total blood volume.

Still referring to the exemplary deployment including 1229 data recordings, reference standard blood potassium results ranged from 2.5 to 6.4 mEq/L, with the median value being 4.2 mEq/L. Reference Hct values ranged from 18 to 48 percentage points, with the median value being 34 percentage points. The XGBoost model classified potassium imbalance-defined as either hypokalemia (K+<3.5 mEq/L) or hyperkalemia (K+>5.2 mEq/L) with a total weighted recall of 86%. The precision (also known as the positive predictive value) of the model was 86%, indicating that the model achieved both high sensitivity and a low rate of false positives. The XGBoost classification results are summarized in Table 1 below.

TABLE 1
Performance summary of wearable biosensor
system for identifying potassium imbalance.

| | Precision | Recall | F1-Score |
|---|---|---|---|
| Hypokalemia (<3.5 mEq/L) | 0.85 | 0.93 | 0.89 |
| Normal K+ | 0.88 | 0.90 | 0.89 |
| (≥3.5 mEq/L, ≤5.2 mEq/L) | | | |
| Hyperkalemia (>5.2 mEq/L) | 0.76 | 0.53 | 0.62 |
| Weighted Accuracy | 0.86 | 0.86 | 0.86 |

In summary, the evaluation embodiments, and the illustrative example, described above involve a non-invasive potassium status classification machine-learning algorithm using a reflected or refracted light corresponding to a PPG sensor that is either coupled to a skin surface of a patient or disposed under the skin surface. The algorithm used by the sensor to classify hyperkalemia, hypokalemia or normal potassium levels was derived from optical sensor features. In some use cases such as the embodiment described above, the machine learning algorithm for detecting potassium imbalance is derived from a sophisticated extension of photoplethysmography (PPG). PPG is an optical sensing technique that measures volumetric variations in blood flow caused by the systole-diastole cardiac cycle. When a pathology disrupts that cycle, as with potassium imbalance, the resulting changes in photon absorption and reflectance can be detected in the PPG waveform. The PPG sensor module contains multiple optical paths arranged in a specific geometric configuration that has been specifically configured for use in hemodynamic monitoring of blood vessels, arteriovenous fistulas, or homogenously perfused tissue sites.

One of the other key physiologic parameters measured by the sensor is hematocrit (Hct). The association between PPG signals and hemoglobin concentration has been shown repeatedly, which demonstrate a non-linear relationship between changes in Hct and serum potassium. For example, this relationship was corroborated by the reference blood results obtained in the deployment discussed above. As has also been demonstrated, an inverse correlation exists between erythrocyte potassium concentration and Hct. Thus, a decrease in Hct is compensated by an increase in the erythrocyte potassium concentration. As has been further demonstrated, a quadratic relationship exists between whole blood potassium and Hct, represented as a polynomial curve. In order to optimize sensor data quality and machine learning algorithm performance, the PPG sensor module was augmented with a three-axis accelerometer and a temperature sensor.

In further discussion of the deployment discussed above, by placing the wearable biosensing device directly over a blood vessel, arteriovenous fistula, or homogenously perfused tissue site, the wearable biosensing device is uniquely positioned to capture PPG signal data over a relatively short period of interrogation time. This signal data is then processed via an Extreme Gradient Boosting (XGBoost) classification model to classify the signal as either hypokalemia (K+<3.5 mEq/L), hyperkalemia (K+>5.2 mEq/L) or normal potassium (3.5 mEq/L≤K+≤5.2 mEq/L), as noted above. This signal processing can then be repeated at precise intervals to enable the development of high-fidelity K+ classification trend lines. The trend lines could provide an opportunity for the data processing system 140 to track and follow the chronological changes in a patient's potassium levels across multiple dialysis sessions and interdialytic periods.

The combination of a wearable biosensing device that is configured to be worn continuously, directly over a blood vessel, hemodialysis arteriovenous fistula, graft, or homogenously perfused tissue site, and the development of a highly accurate potassium classification model enables automated potassium control for patients receiving hemodialysis for end-stage kidney failure, especially in between hemodialysis sessions when significant hyperkalemia can arise without symptoms. Of course, the classification and control of potassium is in addition to the near continuous measurement of Hct and other metrics.

Current potassium and hemoglobin management strategies for hemodialysis patients are based upon dietary modification, renal anemia treatments and tailored hemodialysis therapy. However, routine monitoring of these treatments is often based upon pre- and post-dialysis blood tests, typically once per month. However, in contrast, embodiments of the disclosure enable a near continuous method of monitoring potassium levels that enables personalized care for each patient, during and between hemodialysis sessions, by promoting adherence to potassium restricted diets and more timely adjustment of their medications and dialysis therapy.

A current alternative for non-invasive potassium classification involves the use of 12-lead and single-lead electrocardiograms (ECG). However, these methodologies are less accurate than the embodiments utilizing the wearable biosensing device and machine learning algorithms described herein. Additionally, current potassium and hemoglobin management strategies are not currently integrated with additional physiologic parameters such as skin temperature, heart rate and hematocrit. It should be noted that some embodiments of the wearable biosensing device include a single-lead ECG sensor to augment the PPG-only approach described above.

It should be appreciated that the machine learning techniques utilized in connection with the biosensing device described above enable non-invasive detection of potassium imbalance in a group of patients receiving hemodialysis. Such technology enables remote and continuous monitoring of potassium imbalance in a wide variety of clinical circumstances, including for many patients with advanced chronic kidney disease and those with end-stage kidney disease between in-center or home dialysis session.

Referring now to FIG. 7B, a flow diagram illustrating operations performed by the system of FIG. 6 to train and apply a machine learning model to generate a classification of a potassium imbalance within a patient is shown in accordance with some embodiments. Each block illustrated in FIG. 7B represents an operation performed in the method 720. It should be understood that not every operation illustrated in FIG. 7B is required. In fact, certain operations may be optional. The discussion of the operations in FIG. 7B may be done so with reference to the components illustrated in other figures or otherwise described herein.

The method 720 begins when the data processing system 140 of FIG. 1 obtains historical data on which to train a machine learning model with the historical data including at least patient data 722a (e.g., demographic data, age data, etc.), biosensing data 722b (e.g., PPG signal data), and diagnostic data 722c (e.g., bloodwork results) (collectively, “historical data 722”). It should be understood that the historical data 722 illustrated in FIG. 7B refers to components of the obtained data 202 illustrated in FIG. 2. Example historical data may include, for a set of patients undergoing hemodialysis treatments, results of blood draws that were performed within, for example, 15 minutes of the beginning, or end of the dialysis session, with a mean time of seven minutes between the pre-dialysis blood draw and the beginning of the session and a mean time of five minutes between the end of the session and the post-dialysis session. Additionally, the historical data may include biosensing data was obtained for each patient, within, for example, five minutes of these blood draws.

From the historical data 722, a trained machine learning model is generated by processing obtained data (e.g., historical data 722) using a machine learning algorithm (block 724). As discussed above in detail, embodiments of the disclosure may implement different artificial intelligence or machine learning techniques such the training of a classification algorithm or neural network. As one example, a machine learning model is generated as a classification model trained and configured to distinguish between patients with hypokalemia (K+<3.5 mEq/L), hyperkalemia (K+>5.2 mEq/L) and normal potassium levels (3.5-5.2 mEq/L).

Following the training of the machine learning model, the method 720 continues by obtaining at least current patient data (e.g., demographic data, age gender, etc.) and biosensing data from a current patient (“current data”) (block 726). The trained machine learning model is then applied to the current data resulting in a set of machine learning results that classify a serum potassium level of the patient (block 728), which includes one or more of predictions of metric data 730a, a patient health status 730b, and/or any phase result of a risk stratification 730c (as discussed above). For instance, the classification prediction may include a classification of the serum potassium levels as hyperkalemia (>5.2 mEq/L), hypokalemia (<3.5 mEq/L) or normokalemia (3.5-5.2 mEq/L), where the prediction classification is either provided as a patient health status 730b or a risk stratification 730c.

The set of machine learning results (730a-730c) may be provided in one or more graphical user interfaces (GUIs) (block 732), which may include, for example, a patient portal and/or a clinician portal. Based on the classification prediction provided by the machine learning model, one or more alerts may be provided within the GUIs and/or via a network message such as a text message or email.

### b. Example Use Case—Signal Quality and Hb/Hct Quantification

In a second illustrative example, the data processing system 140 deploys a set of machine learning models, each trained and configured for a particular purpose described below that contribute to identifying patients with hypo- or hyperkalemia. The identification of patients with hypo- or hyperkalemia relies on high quality sensor data and an accurate hematocrit measurement. The second illustrative example describes the methodology performed by the data processing system 140 for discriminating sensor reads of the biosensing device 100 based on signal quality, quantifying hematocrit (Hct) and/or hemoglobin (Hb) resulting in improved identification of patients with hypo- or hyperkalemia.

This methodology includes obtaining measurements of multiple critical parameters for monitoring the general health, vascular access patency, and fluid status of patients undergoing hemodialysis, which enables the improvement of clinical outcomes, reduces mortality, and lowers healthcare costs by preventing vascular access failure, electrolyte imbalance induced arrhythmias, and fluid-overload episodes requiring hospitalization. This is accomplished through the monitoring of a patient's vascular access health and fluid status to minimize clinic visits and enable early intervention for the sequelae of end-stage renal disease (ESRD).

The methodology includes a first sub-methodology of identifying high quality measurements captured by the biosensing device 100 (in contrast to low quality measurements) and a second sub-methodology involving deployment of multiple machine learning models configured to assess Hct, Hb, and dyskalemia. The first sub-methodology of identifying high quality measurements captured by the biosensing device 100 is illustrated in FIG. 7C and discussed below.

Referring now to the second sub-methodology, the quantification of Hct and/or Hb is based on a transfer function fitting a PPG parameter referred to as the amplitude response ratio (R) of two LED-photodiode pairs, which may be included as part of the PPG sensor logic 455 of FIG. 4. The two LEDs may both emit light within the near infrared light range (e.g., 800 to 2,500 nm, and in some embodiments, at approximately 940 nm), and may both be positioned at different distances from the same photodiode, which may also be included in the PPG sensor logic 455. The parameter R is fit to Hct and Hb by a second-order transfer function dependent on the geometry of the optical sensor module, as disclosed in U.S. Pat. Nos. 11,045,123 and 11,406,274. Specifically, this transfer function is dependent on the effective optical path length of the sensor module, which is determined by the absorption and scattering behavior of the tissue of interest and the relative positions of the source-detector pairs that comprise the sensor module.

The data processing system 140 identifies patients with hypo-, normo-, or hyperkalemia, e.g., as defined in Table 2, using features from multiple optical channels and a three-axis accelerometer as its input vector. Classification performance (sensitivity and specificity) is varied through adjustment (in some embodiments, optimization) of the hyperparameters of the models, where the hyperparameters ensure that a model runs efficiently, makes accurate predictions, and is not susceptible to overfitting (i.e., significantly worse performance on unseen data compared to training data). The classification performance of a model may also be improved through re-training the model on new data. In embodiments, these adjustments, improvements, or optimizations are performed in accordance with Good Machine Learning Practices (GMLP).

TABLE 2
Ranges of serum potassium concentration indicating
presence and type of dyskalemia.

| Potassium Status | Range (mEG/L) |
|---|---|
| Hypokalemia | <3.5 |
| Normal | 3.5-5.2 |
| Hyperkalemia | >5.2 |

The biosensing device 100 is deployed to obtain measurements (readings) for each of hematocrit (Hct), hemoglobin (Hb), and potassium (K+) at regular intervals. The readings are then stored in a de-identified manner on the memory 420 of FIG. 4 or on storage of or associated with the data processing system 140 (e.g., storage of the local hub 130 or of cloud computing resources accessible via the network 170). Each reading corresponds to a single contiguous sampling of the one or more sensors of the biosensing device 100 over a set or predetermined time period (e.g., 15, 30, 45, 60 seconds, etc.). Each reading is further is associated with a unique, arbitrary numeric tag representing the patient wearing the biosensing device 100, where the tag is created automatically when a given patient is added to the system and ensures that the data from that patient—e.g., both reference data and that which is generated by the data processing system 140—can be collected and analyzed in concert. Additionally, during the algorithm development process, the unique tag also enables each patient to be pre-assigned to the training, test, or validation data subsets and ensures that data from that patient does not appear in more than one of those subsets.

In some embodiments, upon capture by the biosensing device 100, the raw sensor data is stored redundantly in cloud storage (e.g., private cloud) and a document database, where recorded raw sensor data are associated with (e.g., append to data, store as a key/value pair, etc.) a unique ‘ObjectID’ linking each recorded raw sensor reading to the original raw data record. As a result, the analyses performed on the raw sensor data (and subsequent metrics) are traceable back to a binary file that was originally written by the biosensing device 100 upon capture of the readings. This approach means that verification and validation testing of all analyses is performed by scripts that re-run the entire data processing and analysis pipeline, with no manual input permitted.

Additionally, it should be noted that the raw data sensor readings captured by the biosensing device 100 taken during dialysis may be excluded from the analyses discussed herein. Specifically, the removal of fluid from a patient body during dialysis has a significant effect on a patient's true Hb, Hct, and potassium levels. In some embodiments, only reads captured within a relatively narrow window of time on either side of a blood draw will be included in any process that aims to improve the predictive accuracy of these machine learning models below as the readings may then be compared to the blood draw analysis results to ensure accuracy of the readings.

Referring now to FIG. 7C, a flow diagram illustrating operations performed by the system of FIG. 6 to identify high quality measurements captured by the biosensing device 100 and detection of metrics through the use of machine learning techniques is shown in accordance with some embodiments. Each block illustrated in FIG. 7C represents an operation performed in the method 740. It should be understood that not every operation illustrated in FIG. 7C is required. In fact, certain operations may be optional to complete the method 740. The discussion of the operations in FIG. 7C may be done so with reference to the components illustrated in other figures or otherwise described herein.

The method 740 begins when the data processing system 140 of FIG. 1 acquires biosensing data (block 742). The acquisition of the biosensing data may refer to the capturing of data such as specific reflected or refracted light signals as discussed herein by a biosensing device 100 or when the data processing system 140 obtains such from the biosensing device 100. Following acquisition of the biosensing data, the data processing system 140 determines whether a temperature reading captured as part of the biosensing data satisfies a skin temperature threshold comparison (block 744). The temperature reading corresponds to the temperature of the skin of the patient as captured by the biosensing device 100. In one embodiment, the skin temperature threshold comparison may determine whether the temperature is greater than 26.5° C. In other embodiments, the temperature threshold comparison may seek to determine whether the temperature reading is within a predetermined threshold temperature range. In some instances, a failure to satisfy the temperature threshold comparison indicates that the biosensing device 100 is not properly adhered or coupled to the patient's skin; thus, the quality and accuracy of the biosensing data is in doubt. When the temperature threshold comparison is not satisfied, the biosensing data (reading) is rejected (block 746) and not stored for evaluation by one or more machine learning models to compute Hct and/or Hb, or identify instances of dyskalemia.

When the temperature threshold comparison is satisfied, the biosensing data is then processed by the data processing system 140 such that PPG and accelerometer features are extracted therefrom (block 748). A PPG feature may refer to PPG waveforms (or reflected or refracted light) and an accelerometer feature may refer to motion data, which may be periodic (due to blood flow through the vessel) or random (due to motion noise).

Following extraction of the accelerometer features, the data processing system 140 determines whether the accelerometer features satisfy a motion threshold comparison (block 750). More specifically, the operations of block 750 determine whether the biosensing data was captured during a time while patient wearing the biosensing device 100 was partaking in moderate or vigorous exercise and reject such data. In some instances, the motion threshold comparison operation includes deployment of a random forest classification (RFC) model by the data processing system 140. In such instances, the RFC model is configured to evaluate an input vector comprised of features (readings) from multiple optical channels and a three-axis accelerometer. The output of the RFC model may be a classification prediction indicating whether the reading was captured by the biosensing device 100 when the patient was partaking in moderate to vigorous exercise. When the classification indicates low motion (i.e., that the patient was not partaking in moderate to vigorous exercise when a reading was captured), the classification is understood to satisfy the motion threshold comparison. However, when the classification does not indicate low motion, the classification is understood as not satisfying the motion threshold comparison.

When the motion threshold comparison is not satisfied, the read is rejected (block 746). However, when the motion threshold comparison is satisfied, the data processing system 140 determines whether heartbeats are present within the optical sensor signals of the reading (block 752).

The operations of block 752 include an autocorrelation analysis performed by the data processing system 140 that includes the identification and isolation of valid heartbeats from a bandpass-filtered PPG waveform. First, the PPG signals are preprocessed by down-sampling and applying a bandpass filter, such as a finite impulse response (FIR) filter. The parameters of the bandpass filter may be adjusted based on the detected pulse rate of each data recording, so as to preserve the same waveform features regardless of the patient's pulse rate, which can vary significantly even in healthy subjects. This processed waveform is analyzed using autocorrelation to identify individual heartbeats and identify features of interest. Autocorrelation refers to assessing the similarity of the optical signal waveform with itself at different time lags, the period of which corresponds to the inverse of the pulse rate. These heartbeats may be further discretized into features, such as amplitude or the location of certain fiducial points, the values of which may be compared across the entire waveform to assess the quality of the optical signal waveform. Using this autocorrelation approach, if a certain number of heartbeats are not detected in the PPG channel of a reading, the reading is rejected (block 746). As used herein, a PPG channel refers to an individual source-detector pair.

In some embodiments, machine learning techniques may be utilized to determine (in some instances, optimize) parameters. A first example includes the use of the K-nearest neighbors algorithm to define metaparameter thresholds for identifying canonical heartbeats in a PPG waveform. A second example includes the use of a support vector machine (SVM) model. It should be understood that the operations of blocks 744-752 may be performed in orderings that differ from that should in FIG. 7C (e.g., blocks (i) 744, (ii) 748-750, and (iii) 752 may be performed in serial in any order, in parallel, or concurrently (at least partly overlapping in time)).

Upon successfully satisfying the determinations of block 744, 750, and 752, the data processing system deploys one or more machine learning models to classify the patient with hypo-, normo-, or hyperkalemia (e.g., indicative of the patient's levels of serum potassium) and/or determine Hct and/or Hb metrics of the patient (block 754). The classifications (machine learning results) may be stored in cloud storage and/or local storage.

### c. Example Use Case—Machine Learning for Precision Medicine

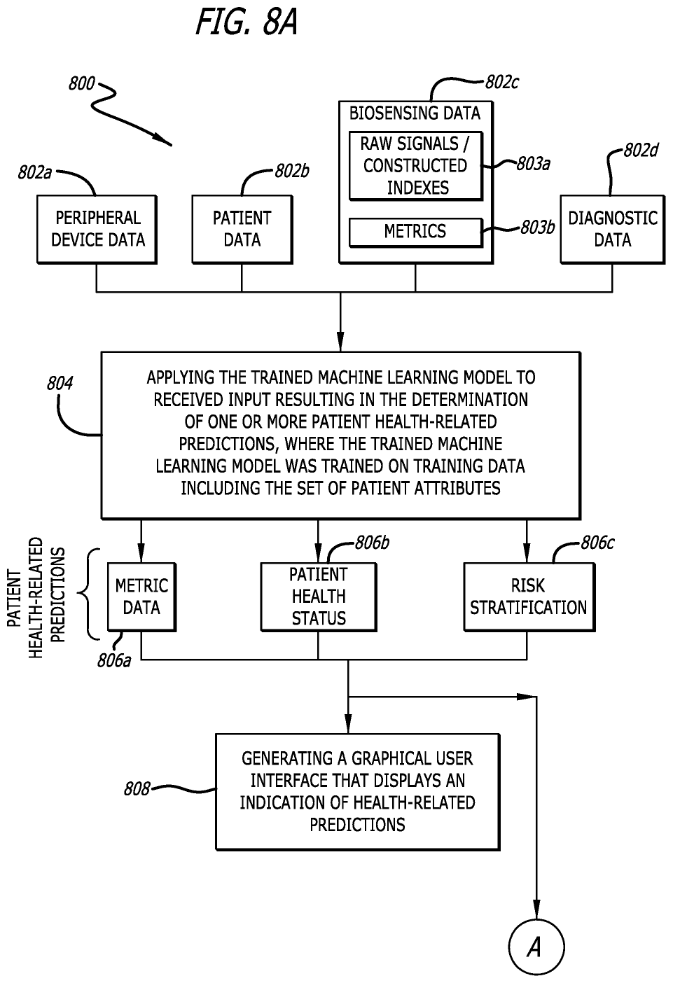

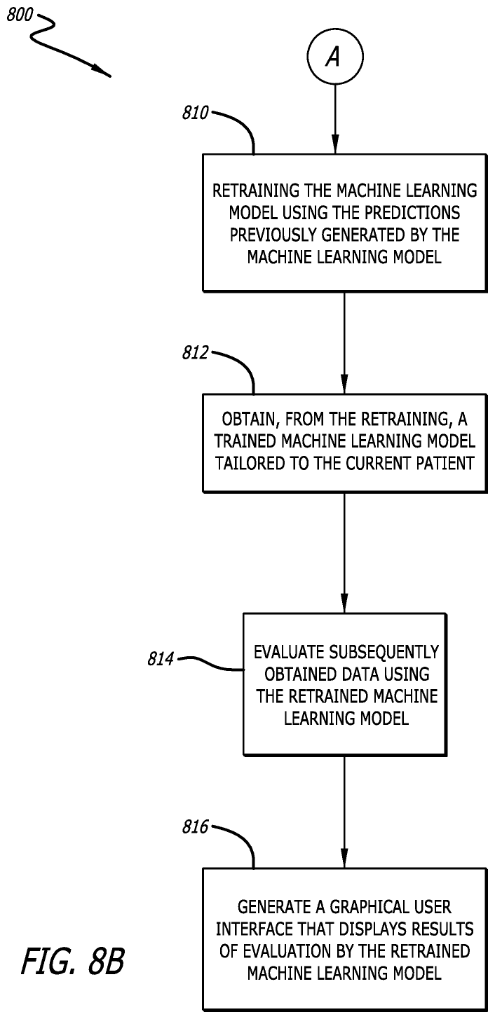

Referring now to FIGS. 8A-8B, a flow chart illustrating operations performed by the system of FIG. 6 to apply a machine learning model to data obtained by a wearable biosensing device and external devices to generate a prediction of the health status of a patient and personalizing a machine learning model for a particular patient through iterative retraining using data obtained by a biosensing device worn by the patient is shown in accordance with some embodiments. The personalization of a machine learning model for a particular patient may be referred to as personalized medicine or precision medicine. Each block illustrated in FIGS. 8A-8B represents an operation performed in the method 800. It should be understood that not every operation illustrated in FIGS. 8A-8B is required. In fact, certain operations may be optional to complete aspects of the acquiring sensor modalities and processing sensor data. The discussion of the operations in FIGS. 8A-8B may be done so with reference to the components illustrated in other figures or otherwise described herein.

Referring to FIG. 8A, the method 800 begins when the processing system 140 of FIGS. 1A-1B obtains information such as peripheral device data 802a, patient data 802b, biosensing data 802c, or diagnostic data 802d (collectively, “obtained data 802”). It should be understood that the obtained data illustrated in FIG. 8A refers to the same obtained data illustrated in FIG. 2. It should be understood that in FIGS. 8A-8B, the obtained data 802 represents current data as opposed to historical data (e.g., previously obtained data).

A trained machine learning model is then applied to the obtained data 802 resulting in a determination of predictions of one or more of metric data 806a, a patient health status 806b, and/or any phase result of a risk stratification 806c (“patient health-related predictions”) (block 804). The feature set on which the machine learning was trained may have included patient attributes, e.g., at least a portion of the patient data 802b and/or diagnostic data 802d.

For example, patient attributes may include gender assigned at birth, gender following hormonal treatments/surgery, diagnosed medical conditions, past cancer diagnoses, medications currently being taken, age, height, weight (and/or historical weights), race/ethnicity, skin pigmentation, residence location (e.g., city, state, country), socio-economic indicators (e.g., education level, household income), whether the patient is/was a smoker or tobacco user (and if so, frequency), whether the patient is/was a drinker (and if so, frequency). From the residence location, an altitude may be determined and included as a patient attribute as altitude affects the hemoglobin and red blood cell indices. For example, hemoglobin (Hb) concentration (g/dL) has been found to be higher in both men and women residing at higher altitudes than those residing at lower altitudes.

In some embodiments, the diagnostic data 202d (or any other aspect of the obtained data 202) may be obtained through patient activation monitoring, which may include automated monitoring of insurance reimbursements. The inclusion of patient attributes specific to an individual patient in the training of the machine learning model serves multiple purposes and provides advantages for the patient over utilizing fewer personalize features. For example, the trained model will be fit closer to relevant data as opposed to fit to data that may pertain to patients of a different gender and/or vastly different height/weight and diagnosed medical conditions. In such situations, a narrowly fit or tailored machine learning model is more personalized to the individual patient. As a result, the training results in coefficients or weighting/biasing configured to more accurately predict the patient's health status and metric data, as well as clinical recommendations, a risk stratification, and dialysis treatment parameters used in generation of executable dialysis machine instructions.

The data processing system 140 may be configured to provide varying levels of personalized patient health-related predictions. More specifically, depending on the historical data utilized in training one or more machine learning models or neural networks, a machine learning model or a neural network may be semi-personalized, meaning training may have included age, gender, current medical conditions, geographic locations, etc. However, in some embodiments, a machine learning model or a neural network may be personalized meaning that, in addition to the parameters referenced with respect to semi-personalized, training may be retrained over time with the patient's own data (“obtained data 202”) so that the machine learning model or neural network becomes more tailored to the individual over time. As is noted above, the patient's own data—i.e., obtained data 202—may include any of peripheral device data 202a, patient data 202b, biosensing data 202c, or diagnostic data 202d collected over time. Further, the processing system 140 may generate one or more graphical user interfaces that illustrate the machine learning results 806a-806c and are configured to display on network devices of the patient and/or a clinician for example (block 808).

Referring now to FIG. 8B and following determination of the machine learning results (806a-806c), the machine learning model may be “retrained” (e.g., trained using revised or updated training data) with additional patient-related data (e.g., captured biosensing data, additional peripheral data, updated/additional patient data, and/or updated/additional diagnostic data) resulting in a larger percentage of the training data being comprised of historical data specific to the patient (block 810). Additionally, the training data may include the predictions previously generated by the machine learning model (e.g., 806a-806c), and optionally with additional reference data, included as part of the training data along with the historical data 802 originally used in training and referenced above, which may include peripheral device data 802a, patient data 802b, biosensing data 802c, or diagnostic data 802d. As a result, the retrained machine learning model will be further tailored (or fit) to the particular patient (block 812).

The retrained machine learning model may then be deployed to evaluate subsequently obtained data to provide patient health-related predictions (block 814). As in block 808, the processing system 140 may generate one or more graphical user interfaces that illustrate the patient health-related predictions (806a-806c) following evaluation by the retrained machine learning model and that are configured to display on network devices of the patient and/or a clinician for example (block 816).

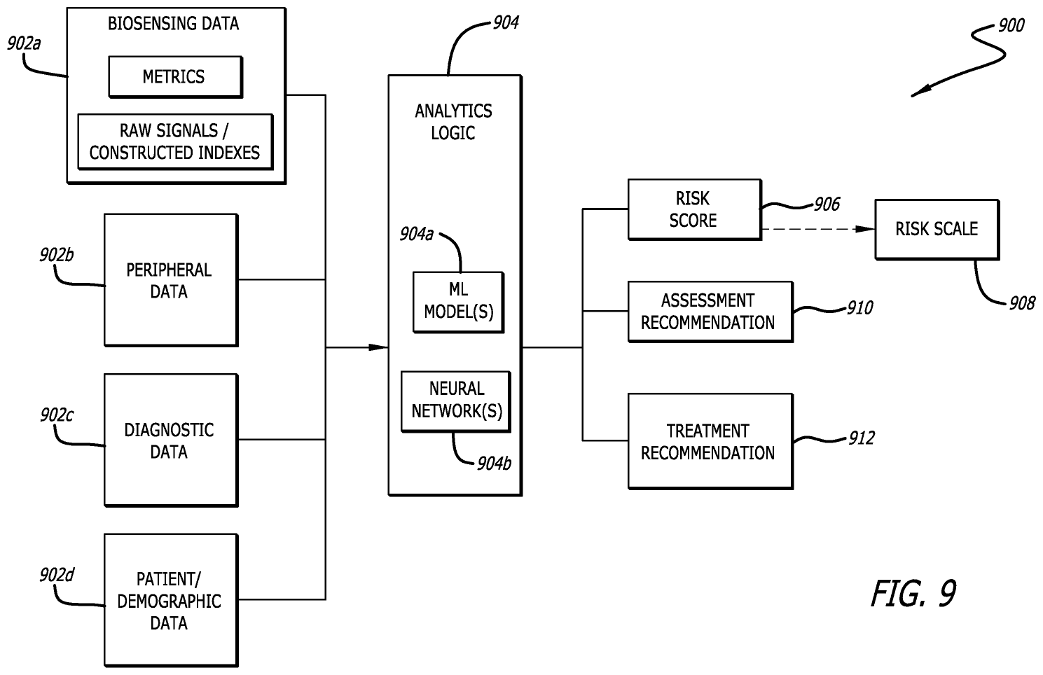

Referring to FIG. 9, a logical flow diagram illustrating exemplary processing flows within an analytics logic resulting in one or more of a risk score determination, an assessment recommendation, a treatment recommendation for a clinician or patient, and/or dialysis treatment parameters that may be provided to a dialysis machine instruction generation logic is shown in accordance with some embodiments. The method 900 illustrates the flow of data obtained by a processing system (i.e., as such as processing system 140 of FIGS. 1A-1B) through an analytics logic 904 (i.e., representative of the analytics logic 616 of FIG. 6). Further, the method 900 may be understood to provide a logic flow of the operations 706-708 of FIG. 7A to generate the machine learning results (710a-710c), and the operations 804-806 of FIG. 8A including receiving the obtained data 802 and applying a trained machine learning model resulting in generation of the patient health-related predictions (806a-806c) and the dialysis machine parameters (806a).

FIG. 9 illustrates that the biosensing data 902a, peripheral device data 902b, diagnostic data 902c, and patient data 902d (collectively, “obtained data 902”) is provided to the analytics logic 904 where one or more analyses performed by the analytics logic 904 results in one or more of a risk score 906 or a risk scale 908, an assessment recommendation 910, or a treatment recommendation (e.g., provided to a clinician, a patient, or a heath-proxy) 912. The processing performed by the analytics logic 904 may include evaluation of the obtained data 902 be either of the machine learning models 904a and/or the neural networks 904b, which are described in detail above.

### VI. Exemplary Graphical User Interfaces

Currently, there is no availability for a patient and/or clinician to visually assess and track predicted metrics of a patient that are determined through analysis of at least biosensing data using trained machine learning models. As seen in FIGS. 1A-1B, systems described herein provide a biosensing device 100 worn by or otherwise coupled to a patient 110 with the biosensing device 100 being communicatively coupled to a data processing system 140 that is configured to deploy machine learning models resulting in patient health-related predictions including metric data, a patient health status, a clinical recommendation, a risk stratification, etc. The patient health-related predictions may then be provided to one or more portals such as a patient portal 180 or a clinician portal 181, where these portals may display the same data but be accessed using different authentication data (e.g., a patient portal 180 corresponds to use of the patient authentication information and a clinician portion 181 corresponds to use of the clinician authentication information).

Thus, at a high level, the graphical user interfaces (GUIs) illustrated in FIGS. 10A-11 provide a technological advantage over current systems merely by enabling a patient or clinician to visualize such patient health-related predictions. Currently, in order to determine a patient's hemoglobin level for example, a clinician is required to have the patient undergo a blood draw, and have the blood analyzed. Differently, the GUIs of FIGS. 10A-11 provide predicted metrics generated from at least biosensing data using machine learning techniques. Additionally, the GUIs illustrated in FIGS. 10A-11 enable a patient or clinician to dynamically alter a view of predictions for a particular metric over time thereby providing an immediate and instantaneous view of the patient's predicted metric over an expanded or contracted period of time. Such is not available with results of a single, traditional blood draw. Further, by providing display screens of multiple metrics in a vertical (or alternatively, horizontal) arrangement, patterns may be quickly deduced in certain situations such as determining a correlation between hemoglobin and hematocrit levels for example.

Referring now to FIGS. 10A-10B, an illustration of a graphical user interface configured for display in a web browser and displaying results of analytics performed by the system of FIG. 6 is shown in accordance with some embodiments. The illustrations provided in FIGS. 10A-10B may represent a portal accessible by a patient (wearer of a biosensing device) and/or a clinician via the web browser 1000. The portal may be comprised of several display portions 1002, 1018, 1034, 1042, and 1052, where a display portion corresponds to a display of data pertaining to a particular metric or analysis.

For example, the display portion 1002 is configured to display certain information pertaining to a patient's hemoglobin levels over time. The display portion 1002 includes an indication as to a predicted current value of the patient's hemoglobin level. As noted above, the biosensing device 100 obtains certain energy measurements (e.g., light, acoustic, and/or pressure) through particularized hardware sensors, where the energy measurements are analyzed through machine learning techniques in order to generate predictions and/or classifications of metrics, a patient health-status, and/or treatment recommendations. Referring again to display portion 1002, the predicted hemoglobin level 1004 may be a result of processing biosensing data by a trained machine learning model as discussed above. Further, the display portion 1002 includes one or more time adjustment user interface (UI) elements 1006, 1008 that are configured to receive user input corresponding to an adjustment of time over which predicted hemoglobin levels are displayed in the graphical display 1010. Thus, by providing user input to either of the UI elements 1006, 1008, the content displayed in the graphical display 1010 may be altered by either expanding or contracting the time period for which predicted hemoglobin data is shown. The graphical display 1010 includes a target area 1012 (e.g., shaded or displayed in a visually distinct manner compared to the rest of the graphical display 1010), where the target area 1012 corresponds to a target range for hemoglobin levels for the patient. Finally, the display portion 1002 includes points 1014 corresponding to a predicted hemoglobin level at a particular point in time, where the set of points 1016 refers to a set of consecutive points (e.g., no read rejections or readings taken during dialysis).

The display portion 1018 pertains to predicted hematocrit levels includes many of the same features as the display portion 1002 including a current predict metric level 1020, time adjustment UI elements 1022, 1024, a graphical display 1026 of points 1030 over time as well as a target area 1028 and a set of consecutive points 1032. It is noted that not all display portions will include any data points and/or a set of consecutive points.

With reference to FIG. 10B, the display portion 1034 provides a graphical display 1040 of the data available for viewing by through the portal and includes time adjustment UI elements 1036, 1038 that are configured to receive user input to expand or contract the time period shown. For instance, expanding the time period by sliding the left circular icon of the time adjustment UI 1038 may “zoom out” the time period such that a time period of, for example, December 1-December 11 is shown. Additionally, the display portion 1042 pertains to predicted potassium levels and includes many of the same features as the display portion 1002 including time adjustment UI elements 1044, 1046, a graphical display 1048 of abnormal potassium readings (e.g., reading 1050) over time. As with the points 1014, 1030 shown in the display portions 1002, 1018, respectively, the abnormal potassium readings are a result of the deployment of a machine learning model that is configured to receive particular features extracted from at least biosensing data (and optionally other data as discussed above) to predict a potassium level of the patient.

Finally, the display portion 1052 pertains to skin temperature readings and includes many of the same features as the display portion 1002 including time adjustment UI elements 1056, 1058, a graphical display 1060 of skin temperature readings 1062 over time.

FIG. 11 provides an illustration of a graphical user interface configured for display via an application processing on a network device and displaying results of analytics performed by the system of FIG. 6 in accordance with some embodiments. The illustration FIG. 11 may represent the display of a dedicated software application (“app”) that is processing on a network device such as a mobile phone or tablet, and configured for display on a hardware screen 1102. The app may include a display portion 1104 corresponding to a metric (such as any of those displayed in FIGS. 10A-10B) or others, such as heart rate as shown in FIG. 11. The display portion 1104 includes many of the same features as the display portion 1002 of FIG. 10A including a current heart rate reading 1106, time adjustment UI elements 1108, 1108, and a graphical display 1112 of skin temperature readings 1114 over time.

One particular embodiment of the disclosure is directed to a system configured to determine patient health-related predictions of a first patient, the system comprising a biosensing device configured to be mounted on skin of the first patient, the biosensing device including a processor, a power source, memory, and at least one sensing element, wherein the at least one sensing element is configured to capture an energy measurement, and a non-transitory computer readable medium having logic stored thereon that, when executed by one or more processors, is configured to cause performance of operations including: obtaining, from the biosensing device, the energy measurement, deploying a machine learning model to analyze features of the energy measurement resulting in a first patient health-related prediction, and generating a graphical user interface illustrating the first patient health-related prediction.

In some embodiments, the energy measurement is reflected or refracted light emitted from a light source, and wherein the sensing element is configured (i) emit light from a light source and (ii) capture the reflected or refracted light emitted from the light source after traveling across an optical path with passage to or through a vessel or homogenously perfused tissue site. In some instances, the operations may include: obtaining, from the biosensing device, the reflected or refracted light, and deploying the machine learning model to analyze the reflected or refracted light resulting in the first patient health-related prediction. In some embodiments, the machine learning model is configured to receive as input an input feature vector that includes features corresponding to the reflected or refracted light and additional features corresponding to peripheral device data, patient data, or diagnostic data. In other embodiments, the first patient health-related prediction is one of (i) a prediction of a current or future metric as indicated by the reflected or refracted light, (ii) a prediction of a current or future patient health status, or (ii) a prediction of any of a set of phases of a risk stratification.

In some instances, the machine learning model is either (i) a classification model generated using a classification machine learning algorithm or (ii) a neural network, wherein the machine learning model was trained on historical patient health data of a plurality of patients. In some embodiments, the graphical user interface illustrating the first patient health-related prediction is remotely accessible to the first patient on a first network device and a clinician on a second network device, and wherein the operations further include: comparing the first patient health-related prediction to a first threshold, and based on a result of comparing the first patient health-related prediction to the first threshold, automatically generating and transmitting an alert to each of the first network device and the second device and providing access to a report detailing the first patient health-related prediction and additional results of deploying the machine learning model to the patient health data.

Other implementations may include a method comprising operations corresponding to aspects of the system noted above. Yet further implementations may include a non-transitory computer-readable medium having stored thereon instructions that, when executed by one or more processors, cause the one or more processor to perform operations corresponding to aspects of the system noted above.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A system configured to determine patient health-related predictions of a first patient, the system comprising:
a biosensing device configured to be mounted on skin of the first patient, the biosensing device including a processor, a power source, memory, and at least one sensing element, wherein the at least one sensing element is configured to capture an energy measurement; and
a non-transitory computer readable medium having logic stored thereon that, when executed by one or more processors, is configured to cause performance of operations including:
obtaining, from the biosensing device, the energy measurement,
deploying a machine learning model to analyze features of the energy measurement resulting in a first patient health-related prediction, and
generating a graphical user interface illustrating the first patient health-related prediction.

2. The system of claim 1, wherein the energy measurement is reflected or refracted light emitted from a light source, and wherein the sensing element is configured to (i) emit light from a light source and (ii) capture the reflected or refracted light emitted from the light source after traveling across an optical path with passage to or through a vessel or homogenously perfused tissue site.

3. The system of claim 2, wherein the operations further include:
obtaining, from the biosensing device, the reflected or refracted light, and
deploying the machine learning model to analyze the reflected or refracted light resulting in the first patient health-related prediction.

4. The system of claim 1, wherein the machine learning model is configured to receive as input an input feature vector that includes features corresponding to the reflected or refracted light and additional features corresponding to peripheral device data, patient data, or diagnostic data.

5. The system of claim 1, wherein the first patient health-related prediction is one of (i) a prediction of a current or future metric as indicated by the reflected or refracted light, (ii) a prediction of a current or future patient health status, or (ii) a prediction of any of a set of phases of a risk stratification.

6. The system of claim 1, wherein the machine learning model is either (i) a classification algorithm or (ii) a neural network, wherein the machine learning model was trained on historical patient health data of a plurality of patients.

7. The system of claim 1, wherein the graphical user interface illustrating the first patient health-related prediction is remotely accessible to the first patient on a first network device and a clinician on a second network device, and
wherein the operations further include:
comparing the first patient health-related prediction to a first threshold, and
based on a result of comparing the first patient health-related prediction to the first threshold, automatically generating and transmitting an alert to each of the first network device and the second device and providing access to a report detailing the first patient health-related prediction and additional results of deploying the machine learning model to the patient health data.

8. A method for determining patient health-related predictions of a first patient, the method comprising:
obtaining, from a biosensing device, an energy measurement, wherein the biosensing device is configured to be mounted on skin of the first patient, the biosensing device including a processor, a power source, memory, and at least one sensing element, wherein the at least one sensing element is configured to capture an energy measurement;
deploying a machine learning model to analyze features of the energy measurement resulting in a first patient health-related prediction; and
generating a graphical user interface illustrating the first patient health-related prediction.

9. The method of claim 8, wherein the energy measurement is reflected or refracted light emitted from a light source, and wherein the sensing element is configured to (i) emit light from a light source and (ii) capture the reflected or refracted light emitted from the light source after traveling across an optical path with passage to or through a vessel or homogenously perfused tissue site.

10. The method of claim 9 further comprising:
obtaining, from the biosensing device, the reflected or refracted light, and
deploying the machine learning model to analyze the reflected or refracted light resulting in the first patient health-related prediction.

11. The method of claim 8, wherein the machine learning model is configured to receive as input an input feature vector that includes features corresponding to the reflected or refracted light and additional features corresponding to peripheral device data, patient data, or diagnostic data.

12. The method of claim 8, wherein the first patient health-related prediction is one of (i) a prediction of a current or future metric as indicated by the reflected or refracted light, (ii) a prediction of a current or future patient health status, or (ii) a prediction of any of a set of phases of a risk stratification.

13. The method of claim 8, wherein the machine learning model is either (i) a classification model generated using a classification machine learning algorithm or (ii) a neural network, wherein the machine learning model was trained on historical patient health data of a plurality of patients.

14. The method of claim 8, wherein the graphical user interface illustrating the first patient health-related prediction is remotely accessible to the first patient on a first network device and a clinician on a second network device, and
further comprising:
comparing the first patient health-related prediction to a first threshold; and
based on a result of comparing the first patient health-related prediction to the first threshold, automatically generating and transmitting an alert to each of the first network device and the second device and providing access to a report detailing the first patient health-related prediction and additional results of deploying the machine learning model to the patient health data.

15. A non-transitory computer-readable medium having stored thereon instructions that, when executed by one or more processors, cause the one or more processor to perform operations for determining patient health-related predictions of a first patient, the operations including:
obtaining, from a biosensing device, an energy measurement, wherein the biosensing device is configured to be mounted on skin of the first patient, the biosensing device including a processor, a power source, memory, and at least one sensing element, wherein the at least one sensing element is configured to capture an energy measurement;
deploying a machine learning model to analyze features of the energy measurement resulting in a first patient health-related prediction; and
generating a graphical user interface illustrating the first patient health-related prediction.

16. The non-transitory computer-readable medium of claim 15, wherein the energy measurement is reflected or refracted light emitted from a light source, and wherein the sensing element is configured to (i) emit light from a light source and (ii) capture the reflected or refracted light emitted from the light source after traveling across an optical path with passage to or through a vessel or homogenously perfused tissue site.

17. The non-transitory computer-readable medium of claim 16 further comprising:
obtaining, from the biosensing device, the reflected or refracted light, and
deploying the machine learning model to analyze the reflected or refracted light resulting in the first patient health-related prediction.

18. The non-transitory computer-readable medium of claim 15, wherein the machine learning model is configured to receive as input an input feature vector that includes features corresponding to the reflected or refracted light and additional features corresponding to peripheral device data, patient data, or diagnostic data.

19. The non-transitory computer-readable medium of claim 15, wherein the first patient health-related prediction is one of (i) a prediction of a current or future metric as indicated by the reflected or refracted light, (ii) a prediction of a current or future patient health status, or (ii) a prediction of any of a set of phases of a risk stratification.

20. The non-transitory computer-readable medium of claim 15, wherein the machine learning model is either (i) a classification model generated using a classification machine learning algorithm or (ii) a neural network, wherein the machine learning model was trained on historical patient health data of a plurality of patients.

21. The non-transitory computer-readable medium of claim 15, wherein the graphical user interface illustrating the first patient health-related prediction is remotely accessible to the first patient on a first network device and a clinician on a second network device, and
wherein the operations further include:
comparing the first patient health-related prediction to a first threshold; and
based on a result of comparing the first patient health-related prediction to the first threshold, automatically generating and transmitting an alert to each of the first network device and the second device and providing access to a report detailing the first patient health-related prediction and additional results of deploying the machine learning model to the patient health data.
